(19) United States
(12) Patent Application Publication
(10) Pub. No.: US 2011/0104725 A1
(43) Pub. Date: May 5, 2011

(54) Method of Effecting Coagulation in a Droplet

(75) Inventors: Vamsee K. Pamula, Durham, NC (US); Sitaram Emani, Brookline, MA (US); Allen Eckhardt, Durham, NC (US); Carrie Graham, Raleigh, NC (US); Jeremy Rouse, Raleigh, NC (US)

(73) Assignees: ADVANCED LIQUID LOGIC, INC., Research Triangle Park, NC (US); CHILDREN'S MEDICAL CENTER CORPORATION, Boston, MA (US)

(21) Appl. No.: 12/990,766

(22) PCT Filed: May 4, 2009
(86) PCT No.: PCT/US2009/042699

§ 371 Date: Dec. 8, 2010

Related U.S. Application Data
(60) Provisional application No. 61/049,800, filed on May 2, 2008.
(60) Provisional application No. 61/077,184, filed on Jul. 1, 2008.
(60) Provisional application No. 61/091,817, filed on Aug. 26, 2008.
(60) Provisional application No. 61/101,321, filed on Sep. 30, 2008.

(51) Int. Cl. G01N 33/53

(52) U.S. Cl. 435/7.92

(57) ABSTRACT

The invention provides techniques for coagulating blood on a droplet actuator. The invention also provides methods of manipulating the coagulated blood including a variety of droplet operations that may be conducted using the coagulated blood. Further, the invention provides a variety of assays that make use of the coagulated blood or various blood samples as input.

### 1 RELATED APPLICATIONS

This application claims priority to and incorporates by reference the following U.S. Patent Applications: 61/049,800, entitled “Droplet-Based Coagulation Assays, filed on May 2, 2008; 61/077,184, entitled “Droplet-based coagulation assays, filed on Jul. 1, 2008; 61/091,817, entitled “Droplet-based coagulation assays, filed on Aug. 26, 2008; and 61/101,321, entitled “Droplet actuator techniques using blood, filed on Sep. 30, 2008.

### 2 FIELD OF THE INVENTION

The invention relates to methods and devices for coagulating droplets, such as blood droplets; assessing coagulability in coagulable sample droplets; and performing assays using the same.

### 3 BACKGROUND

Droplet actuators are used to conduct a wide variety of droplet operations. A droplet actuator typically includes two substrates separated to form a droplet operations gap. The substrates include electrodes for conducting droplet operations. The gap between the substrates is typically filled with a filler fluid that is immiscible with the liquid that is to be subjected to droplet operations. Droplet operations are controlled by electrodes associated with one or both of the substrates. There is a need for techniques for working with coagulatable samples, such as blood, on a droplet actuator, such as methods for manipulating and testing a coagulatable sample and/or subcomponents of a coagulatable sample. There is a need for techniques for causing coagulation of a coagulatable sample on a droplet actuator, conducting droplet operations using the coagulated sample or sub-components of the coagulated sample, and/or conducting testing of various components of the coagulated sample.

### 4 BRIEF DESCRIPTION OF THE INVENTION

The invention provides a method of effecting coagulation in source droplet. The method may, in certain embodiments, include: providing an oil medium; providing in the oil medium a source droplet may, in certain embodiments, include one or more coagulatable substances; treating in the oil medium the source droplet to effect coagulation of the one or more coagulatable substances to yield a coagulated droplet in the oil medium may, in certain embodiments, include a coagulated portion and supernatant. The source droplet may, in certain embodiments, include a biological fluid. The biological fluid may, in certain embodiments, include a blood sample. The blood sample may, in certain embodiments, include whole blood. The blood sample may consist essentially of whole blood. The blood sample may consist of whole blood. The blood sample may, in certain embodiments, include one or more natural blood components. The blood sample may, in certain embodiments, include one or more artificial blood components. The blood sample may, in certain embodiments, include one or more anticoagulants. The anticoagulant may, in some embodiments, be selected from the group consisting of coumarines, vitamin K antagonists, acenocoumarol, phenprocoumon, brodifacoum, phenindione, heparins, low molecular weight heparin, synthetic pentasaccharide inhibitors of Factor Xa, and thrombin inhibitors. The one or more artificial blood components may, in some cases, include one or more artificial platelet components and/or one or more artificial oxygen carriers. The source droplet may, in certain embodiments, include a milk sample. The source droplet may, in certain embodiments, include a plant sample (e.g., soy milk). The source droplet may, in certain embodiments, include coagulatable beads.

Treating the source droplet to effect coagulation may, in certain embodiments, include combining the sample droplet with a procoagulant droplet including a procoagulant; contacting the sample droplet with a procoagulant; inclubating the sample droplet for a period of time sufficient to permit coagulation; maintaining the sample droplet in a substantially stationary position for a period of time sufficient to permit coagulation; heating the sample droplet; and/or cooling the sample droplet. In some cases, coagulation is effected in a sample droplet while the sample droplet is being subjected to movement induced by an electrode. In some cases, coagulation is effected in a sample droplet while the sample droplet is being exposed to an electrical field. Treating the source droplet to effect coagulation may, in some embodiments, be accomplished in the presence of an electrical field.

Various embodiments may include conducting an assay using the coagulated droplet as input. The assay may, for example, be affected using droplet operations on a droplet actuator. In some cases, the droplet operations may, for example, be conducted in a droplet operations gap on a droplet actuator. When present, the filler fluid may include an organic oil, such as a silicone oil, an alkane oil, and/or a fluorinated oil. In some cases, the oil medium has a viscosity ranging from about 1 to about 3 cSt. The oil medium may, in some embodiments, be doped with a surfactant. The surfactant may, in certain embodiments, include a linoleic acid based surfactant composition.

The invention provides a reagent droplet with a coagulating amount of a blood coagulant; or an anticoagulating amount of a blood anticoagulant. The droplet actuator further may, in certain embodiments, include a blood droplet. In some embodiments, the droplet actuator includes a substrate; droplet operations electrodes associated with the substrate; one or more dielectric and/or hydrophobic layers atop the substrate and/or electrodes forming a droplet operations surface; and a top substrate separated from the droplet operations surface by a droplet operations gap. The reagent droplet may, in some embodiments, be present in the droplet operations gap and subject to one or more droplet operations mediated by one or more of the droplet operations electrodes. The droplet actuator may also include a blood sample droplet. Droplet operations electrodes may be used to effect droplet operations which result in the combination of the reagent droplet and the blood droplet and thus the coagulation or anticoagulation of the blood droplet. The one or more reagents for quenching coagulation in a blood droplet may, for example, be immersed in an organic filler fluid, such as a silicone oil, an alkane oil, and/or a fluorinated oil filler fluid.

The invention provides a method of conducting a droplet operation using a coagulated droplet. The method may, in certain embodiments, include providing on a droplet actuator a sample droplet including a coagulatable substance. The method may, for example, include inducing or permitting coagulation in the coagulatable sample droplet to yield a coagulated droplet including a supernatant and a coagulated material. The coagulated droplet may be subjected to one or more droplet operations.

The sample droplet may, in certain embodiments, include a biological fluid. The biological fluid may, in certain embodiments, include a blood sample. The blood sample may, in certain embodiments, include whole blood. The blood sample may consist essentially of whole blood. The blood sample may consist of whole blood. The blood sample may, in certain embodiments, include one or more natural blood components. The blood sample may, in certain embodiments, include one or more artificial blood components. The blood sample may, in certain embodiments, include one or more anticoagulants. The one or more anticoagulants may, in some embodiments, be selected from the group consisting of coumarines, vitamin K antagonists, acenocoumarol, phenprocoumon, brodifacoum, phenindione, heparins, low molecular weight heparin, synthetic pentasaccharide inhibitors of Factor Xa, and thrombin inhibitors. The one or more artificial blood components may, in some cases, include one or more artificial platelet components and/or one or more artificial oxygen carriers. The biological fluid may, in other embodiments, include a milk sample or a plant sample. The sample droplet or biological fluid may, in certain embodiments, include coagulatable beads. The coagulated droplet may include coagulated beads. Beads may be coagulated by providing them with cross-linking substances and/or other beads for which the beads have affinity.

The invention includes providing a sample droplet comprising a coagulatable substance on a droplet actuator. This aspect of the invention may, for example, include flowing a blood sample onto a droplet actuator from a subject's circulatory system. In another embodiment, providing a sample droplet comprising a coagulatable substance on a droplet actuator may include flowing a blood sample onto a droplet actuator from a fluid path coupled to an extracorporeal blood circuit. For example, the circuit may include a hemodialysis circuit, hemofiltration circuit, plasmapheresis circuit, apheresis circuit, and/or an oxygenation circuit. The extracorporeal blood circuit may, in certain embodiments, include an extracorporeal membrane oxygenation circuit. The extracorporeal blood circuit may, in certain embodiments, include a cardiopulmonary bypass circuit. The extracorporeal blood circuit may, in certain embodiments, include a cardiac assist device circuit.

The invention includes inducing or permitting coagulation in the coagulatable sample droplet to yield a coagulated droplet comprising a supernatant and coagulated material. The inducing or permitting coagulation may in some cases be effected on a droplet actuator, e.g., in a reservoir on a droplet actuator and/or in a droplet operations gap of a droplet actuator. Coagulation may be induced on the droplet actuator by using droplet operations to combine the sample droplet with a droplet may, in certain embodiments, include a procoagulant. Coagulation may be induced on the droplet actuator by using droplet operations to contact on the droplet actuator the sample droplet with a procoagulant. In other embodiments, coagulation may additionally or alternatively include incubating the sample droplet on the droplet actuator for a period of time sufficient to permit coagulation; retaining the sample droplet in a substantially stationary position on the droplet actuator for a period of time sufficient to permit coagulation; heating the sample droplet on the droplet actuator; and/or cooling the sample droplet on the droplet actuator. Coagulating on a droplet actuator may, in some embodiments, be accomplished in the presence of an electrical field, e.g., while a droplet is being retained in position by a surface tension effect induced by an electrical field. In some cases, an electrical field is used to modulate coagulation. In some embodiments, coagulating may be accomplished while the sample droplet is in contact with the atmosphere (e.g., in the absence of a top plate).

The invention also provides methods of subjecting coagulated droplet to one or more droplet operations. The droplet operation may, in certain embodiments, include an electrode-mediated droplet operation, such as an electrowetting mediated droplet operation and/or a dielectrophoresis mediated droplet operation. The droplet operation may, in various embodiments, include dispensing one or more sub-droplets from the coagulated droplet. The one or more sub-droplets may, in some cases, include one or more sub-droplets substantially lacking coagulated material. The method may include detecting whether the one or more dispensed sub-droplets include one or more sub-droplets substantially lacking coagulated material. The detecting may, in certain embodiments, include visually or optically detecting. The detecting may, in other embodiments, include detecting based on a physical or electrical property of the one or more sub-droplets. The droplet operation may, in various embodiments, include splitting, separating or dividing the coagulated droplet into two or more sub-droplets; transporting the coagulated droplet from one location to another on the droplet actuator; merging or combining two or more droplets, including at least one coagulated droplet, into a single droplet; diluting the coagulated droplet; mixing the coagulated droplet; agitating the coagulated droplet; deforming the coagulated droplet; retaining the coagulated droplet in position; incubating the coagulated droplet, heating the coagulated droplet, and/or cooling the coagulated droplet; disposing of the coagulated droplet; and/or transporting the coagulated droplet out of a droplet actuator.

In some embodiments, inducing or permitting coagulation in the coagulatable sample droplet to yield a coagulated droplet comprising a supernatant and coagulated material may include providing one or more magnetically responsive beads in the coagulatable sample droplet and associating the one or more magnetically responsive beads with the coagulatable material. In some cases, the magnetically responsive beads have affinity for a component of the coagulated coagulatable material. In some cases, at least a portion of the magnetically responsive beads may be physically captured within the coagulated material. A magnet may be used to restrain or substantially immobilize the coagulated material during a droplet splitting or droplet transporting operation to yield a droplet including substantially all of the coagulated material; and a droplet including supernatant and substantially lacking coagulated material.

The invention provides, in certain embodiments, for inducing or permitting coagulation in the coagulatable sample droplet while the sample droplet is in contact with an immiscible filler fluid. The sample droplet may, in some embodiments, be in contact with, or substantially immersed in, an organic filler fluid. The organic filler fluid may, in certain embodiments, include a silicone oil, an alkane oil, and/or a fluorinated oil. The filler fluid may, in certain embodiments, include a surfactant. The surfactant may, in certain embodiments, include a nonionic low hydrophilic-lipophilic balance (HLB) surfactant. The HLB may, in some embodiments, be less than about 10. The HLB may, in some embodiments, be less than about 5. The surfactant may, in some embodiments, be selected from the group consisting of: Triton X-15, Span 85, Span 65, Span 83, Span 80, Span 60, and fluorinated surfactants.

The invention provides a droplet actuator. The droplet actuator may include a reservoir. The reservoir may, in certain embodiments, include an anticoagulant compound. The reservoir may, in certain embodiments, include a coagulatable sample, such as a coagulatable blood sample. The reservoir may include an opening for introducing one or more substances into the reservoir, for example, one or more blood samples may be introduced into the reservoir and combined with one or more anticoagulant compounds to yield an anticoagulated blood component droplet in the reservoir. Thus, the invention provides a droplet actuator comprising a reservoir comprising an anticoagulated blood sample in the reservoir. The anticoagulated blood sample may be subject to droplet operations in a droplet operations gap of the droplet actuator, e.g., by flowing the anticoagulated blood sample in the reservoir through an opening into the droplet operations gap. Alternatively, the reservoir itself may be a virtual or physical reservoir established in the droplet operations gap. The droplet actuator may include electrodes configured for conducting one or more droplet operations using the anticoagulated blood component droplet. The anticoagulant may, in some embodiments, be selected from the group consisting of coumarines, vitamin K antagonists, acenocoumarol, phenprocoumon, brodifacoum, phenindione, heparins, low molecular weight heparin, synthetic pentasaccharide inhibitors of Factor Xa, and thrombin inhibitors.

The reservoir may have a vacuum established therein, e.g., to pull the sample into the reservoir when the reservoir is coupled by a fluid path to a sample source. The anticoagulant may, in some embodiments, be bound to a surface of the reservoir. The surface of the reservoir may, in some embodiments, be heparinized The reservoir may, in certain embodiments, include corn trypsin inhibitor. The reservoir may, in some embodiments, be coupled by a fluid path to a device for collecting a blood sample from a patient's circulatory system. The reservoir may, in some embodiments, be coupled by a fluid path to a device for collecting a blood sample from a central line. The reservoir may, in some embodiments, be coupled by a fluid path to an extracorporeal blood circulation circuit. The extracorporeal blood circulation circuit may, in certain embodiments, include a circuit selected from the group consisting of hemodialysis circuits, hemofiltration circuits, plasmapheresis circuits, apheresis circuits, and/or oxygenation circuits. The extracorporeal blood circulation circuit may, in certain embodiments, include an extracorporeal membrane oxygenation circuit. The extracorporeal blood circulation circuit may, in certain embodiments, include a cardiopulmonary bypass circuit. The extracorporeal blood circulation circuit may, in certain embodiments, include a cardiac assist device circuit. The reservoir may, in some embodiments, be coupled by a fluid path to a sterile hollow needle, e.g., a hollow needle designed for collecting blood in a subject.

As noted, the invention provides a droplet actuator which may include a reservoir with an anticoagulant compound. The reservoir may include an opening for introducing one or more blood samples into the reservoir to yield an anticoagulated blood sample droplet. The droplet actuator may include electrodes configured on one or more substrates for conducting one or more droplet operations using the anticoagulated blood sample droplet. A method of the invention may include subjecting the anticoagulated blood sample droplet to one or more droplet operations mediated by the electrodes. The blood sample may, in certain embodiments, include whole blood. The blood sample may consist essentially of whole blood. The blood sample may consist of whole blood. The droplet actuator may, in certain embodiments, include: a substrate; droplet operations electrodes associated with the substrate; one or more dielectric and/or hydrophobic layers atop the substrate and/or electrodes forming a droplet operations surface; and a top substrate separated from the droplet operations surface by a droplet operations gap. Subjecting the anticoagulated blood sample droplet to one or more droplet operations mediated by the electrodes may, in some embodiments, be executed in the droplet operations gap. One or more of the droplet operations may, in some embodiments, be executed in an organic filler fluid. One or more of the droplet operations may, in some embodiments, be executed in an oil filler fluid. One or more of the droplet operations may, in some embodiments, be executed in a silicone oil, an alkane oil, and/or a fluorinated oil.

The invention provides a method of assessing coagulation in a sample. The method may, in certain embodiments, include providing sample droplets on a droplet actuator, each sample droplet including a blood sample. The method may include quenching coagulation in each droplet to yield quenched droplets. The quenching may, in some embodiments, be effected serially for at least a subset of the sample droplets, such that each droplet in the subset may, in some embodiments, be quenched at a different time relative to other droplets in the subset. The quenching may be effected on a droplet actuator, such as in droplet actuator reservoirs, on a droplet operations surface, or in a droplet operations gap of a droplet actuator. The method may also include analyzing the quenched droplets. For example, the quenched droplets may be analyzed to detect the formation of thrombin-anti-thrombin (TAT) complexes and/or prothrombin fragment F1+2.

Analyzing the quenched droplets may, in certain embodiments, include analyzing the quenched droplets by immunoassay. The immunoassay may, in certain embodiments, include a sandwich ELISA. Analyzing the quenched droplets may, in certain embodiments, include combining on the droplet actuator each quenched droplet with a droplet including beads coated with anti-thrombin antibody. Analyzing the quenched droplets may, in certain embodiments, include splitting on the droplet actuator the coagulated droplet produced to yield a droplet including the beads and a supernatant droplet. Analyzing the quenched droplets may, in certain embodiments, include performing on the droplet actuator a TAT complex assay on the bead-containing droplet. Analyzing the quenched droplets may, in certain embodiments, include performing on the droplet actuator an F1+2 assay on the supernatant droplet.

The TAT complex assay may, for example, include washing on the droplet actuator the beads to provide a first droplet including washed beads. The TAT complex assay may include combining on the droplet actuator the first droplet including washed beads with a droplet including a secondary antibody. The secondary antibody may be labeled with an enzyme (e.g., alkaline phosphatase, horse radish peroxidase, galactosidase, luciferase, etc.) that catalyzes a substrate or it may be labeled with a direct label which can be measured (e.g., fluorophores, nanoparticles, color dyes, etc.). The TAT complex assay may include washing on the droplet actuator the beads including the secondary antibody to provide a second droplet including washed beads. The TAT complex assay may include combining on the droplet actuator the second droplet including the washed beads with an enzymatic substrate, which may, for example, include a chemiluminescence substrate or a fluorescence substrate. The TAT complex assay may include measuring chemiluminescence of the droplet including the chemiluminescence substrate. Various steps of the method may be performed using droplet operations on a droplet actuator.

In certain embodiments, performing on the droplet actuator a TAT complex assay may include combining on the droplet actuator the supernatant droplet with a droplet including F1+2 beads. The method may include washing on the droplet actuator the F1+2 beads to yield a droplet including washed F1+2 beads. The invention may include combining on the droplet actuator a droplet which may, in certain embodiments, include washed F1+2 beads with a droplet including conjugated secondary antibody against F1 and F2. The invention may include combining on the droplet actuator the droplet including washed F1+2 beads and conjugated secondary antibody with an enzymatic substrate, which in some embodiments can be a chemiluminescence substrate or a fluorescence substrate. The invention may include measuring chemiluminescence of the resulting droplet. Various steps of the method may be performed using droplet operations on a droplet actuator. Measuring chemiluminescence may, in some embodiments, be performed on the droplet actuator. In other embodiments, performing on the droplet actuator a TAT complex assay may include depleting the supernatant droplet of TAT complexes through a first ELISA and then performing a second ELISA for F1+2 on the supernatant droplet on the droplet actuator.

Measurements from the assays may be used to calculate TAT complex and F1+2 levels. A system may be provided providing outputs indicative of results of these and other assays. The invention may include determining and outputting a report indicative of lag time to thrombin generation. The invention may include determining total amount of TAT complex or F1+2 generation.

### 5 DEFINITIONS

As used herein, the following terms have the meanings indicated.

“Activate” with reference to one or more electrodes means effecting a change in the electrical state of the one or more electrodes which, in the presence of a droplet, results in a droplet operation.

“Bead,” with respect to beads on a droplet actuator, means any bead or particle that is capable of interacting with a droplet on or in proximity with a droplet actuator. Beads may be any of a wide variety of shapes, such as spherical, generally spherical, egg shaped, disc shaped, cubical and other three dimensional shapes. The bead may, for example, be capable of being transported in a droplet on a droplet actuator or otherwise configured with respect to a droplet actuator in a manner which permits a droplet on the droplet actuator to be brought into contact with the bead, on the droplet actuator and/or off the droplet actuator. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead or one component only of a bead. The remainder of the bead may include, among other things, polymeric material, coatings, and moieties which permit attachment of an assay reagent. Examples of suitable magnetically responsive beads are described in U.S. Patent Publication No. 2005-0260686, entitled, “Multiplex flow assays preferably with magnetic particles as solid phase,” published on Nov. 24, 2005, the entire disclosure of which is incorporated herein by reference for its teaching concerning magnetically responsive materials and beads. The fluids may include one or more magnetically responsive and/or non-magnetically responsive beads. Examples of droplet actuator techniques for immobilizing magnetically responsive beads and/or non-magnetically responsive beads and/or conducting droplet operations protocols using beads are described in U.S. patent application Ser. No. 11/639,566, entitled “Droplet-Based Particle Sorting,” filed on Dec. 15, 2006; U.S. Patent Application No. 61/039,183, entitled “Multiplexing Bead Detection in a Single Droplet,” filed on Mar. 25, 2008; U.S. Patent Application No. 61/047,789, entitled “Droplet Actuator Devices and Droplet Operations Using Beads,” filed on Apr. 25, 2008; U.S. Patent Application No. 61/086,183, entitled “Droplet Actuator Devices and Methods for Manipulating Beads,” filed on Aug. 5, 2008; International Patent Application No. PCT/US2008/053545, entitled “Droplet Actuator Devices and Methods Employing Magnetic Beads,” filed on Feb. 11, 2008; International Patent Application No. PCT/US2008/058018, entitled “Bead-based Multiplexed Analytical Methods and Instrumentation,” filed on Mar. 24, 2008; International Patent Application No. PCT/US2008/058047, “Bead Sorting on a Droplet Actuator,” filed on Mar. 23, 2008; and International Patent Application No. PCT/US2006/047486, entitled “Droplet-based Biochemistry,” filed on Dec. 11, 2006; the entire disclosures of which are incorporated herein by reference.

“Droplet” means a volume of liquid on a droplet actuator that is at least partially bounded by filler fluid. For example, a droplet may be completely surrounded by filler fluid or may be bounded by filler fluid and one or more surfaces of the droplet actuator. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, and various shapes formed during droplet operations, such as merging or splitting or formed as a result of contact of such shapes with one or more surfaces of a droplet actuator. For examples of droplet fluids that may be subjected to droplet operations using the approach of the invention, see International Patent Application No. PCT/US 06/47486, entitled, “Droplet-Based Biochemistry,” filed on Dec. 11, 2006. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Moreover, a droplet may include a reagent, such as water, deionized water, saline solutions, acidic solutions, basic solutions, detergent solutions and/or buffers. Other examples of droplet contents include reagents, such as a reagent for a biochemical protocol, such as a nucleic acid amplification protocol, an affinity-based assay protocol, an enzymatic assay protocol, a sequencing protocol, and/or a protocol for analyses of biological fluids.

“Droplet Actuator” means a device for manipulating droplets. For examples of droplet actuators, see U.S. Pat. No. 6,911,132, entitled “Apparatus for Manipulating Droplets by Electrowetting-Based Techniques,” issued on Jun. 28, 2005 to Pamula et al.; U.S. patent application Ser. No. 11/343,284, entitled “Apparatuses and Methods for Manipulating Droplets on a Printed Circuit Board,” filed on filed on Jan. 30, 2006; U.S. Pat. No. 6,773,566, entitled “Electrostatic Actuators for Microfluidics and Methods for Using Same,” issued on Aug. 10, 2004 and U.S. Pat. No. 6,565,727, entitled “Actuators for Microfluidics Without Moving Parts,” issued on Jan. 24, 2000, both to Shenderov et al.; Pollack et al., International Patent Application No. PCT/US2006/047486, entitled “Droplet-Based Biochemistry,” filed on Dec. 11, 2006; and Roux et al., U.S. Patent Pub. No. 20050179746, entitled “Device for Controlling the Displacement of a Drop Between two or Several Solid Substrates,” published on Aug. 18, 2005; the disclosures of which are incorporated herein by reference. Certain droplet actuators will include a substrate, droplet operations electrodes associated with the substrate, one or more dielectric and/or hydrophobic layers atop the substrate and/or electrodes forming a droplet operations surface, and optionally, a top substrate separated from the droplet operations surface by a gap. One or more reference electrodes may be provided on the top and/or bottom substrates and/or in the gap. In various embodiments, the manipulation of droplets by a droplet actuator may be electrode-mediated, e.g., electrowetting mediated or dielectrophoresis mediated or Coulombic force mediated. Examples of other methods of controlling fluid flow that may be used in the droplet actuators of the invention include devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electroosmotic flow, electrokinetic pumps, ferrofluidic plugs, electrohydrodynamic pumps, attraction or repulsion using magnetic forces and magnetohydrodynamic pumps); thermodynamic principles (e.g. gas bubble generation/phase-change-induced volume expansion); other kinds of surface-wetting principles (e.g. electrowetting, and optoelectrowetting, as well as chemically, thermally, structurally and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causes flow); magnetohydrodynamic forces; and vacuum or pressure differential. In certain embodiments, combinations of two or more of the foregoing techniques may be employed in droplet actuators of the invention. In some embodiments, the droplet actuator is provided as a portable device, permitting analysis at a point of sample collection.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles.

“Filler fluid” means a fluid associated with a droplet operations substrate of a droplet actuator, which fluid is sufficiently immiscible with a droplet phase to render the droplet phase subject to electrode-mediated droplet operations. The filler fluid may, for example, be a low-viscosity oil, such as a silicone oil, an alkane oil, and/or a fluorinated oil. Other examples of filler fluids are provided in International Patent Application No. PCT/US2006/047486, entitled, “Droplet-Based Biochemistry,” filed on Dec. 11, 2006; International Patent Application No. PCT/US2008/072604, entitled “Use of additives for enhancing droplet actuation,” filed on Aug. 8, 2008; and U.S. Patent Publication No. 20080283414, entitled “Electrowetting Devices,” filed on May 17, 2007; the entire disclosures of which are incorporated herein by reference. The filler fluid may fill the entire gap of the droplet actuator or may coat one or more surfaces of the droplet actuator. Filler fluid may be conductive or non-conductive.

“Immobilize” with respect to magnetically responsive beads, means that the beads are substantially restrained in position in a droplet or in filler fluid on a droplet actuator. For example, in one embodiment, immobilized beads are sufficiently restrained in position to permit execution of a splitting operation on a droplet, yielding one droplet with substantially all of the beads and one droplet substantially lacking in the beads.

“Magnetically responsive” means responsive to a magnetic field. “Magnetically responsive beads” include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe₃O₄, BaFe₁₂O₁₉, CoO, NiO, Mn₂O₃, Cr₂O₃, and CoMnP.

“Washing” with respect to washing a magnetically responsive bead means reducing the amount and/or concentration of one or more substances in contact with the magnetically responsive bead or exposed to the magnetically responsive bead from a droplet in contact with the magnetically responsive bead. The reduction in the amount and/or concentration of the substance may be partial, substantially complete, or even complete. The substance may be any of a wide variety of substances; examples include target substances for further analysis, and substances, such as components of a sample, contaminants, and/or excess reagent. In some embodiments, a washing operation begins with a starting droplet in contact with a magnetically responsive bead, where the droplet includes an initial amount and initial concentration of a substance. The washing operation may proceed using a variety of droplet operations. The washing operation may yield a droplet including the magnetically responsive bead, where the droplet has a total amount and/or concentration of the substance which is less than the initial amount and/or concentration of the substance. Examples of suitable washing techniques are described in Pamula et al., U.S. Pat. No. 7,439,014, entitled “Droplet-Based Surface Modification and Washing,” granted on Oct. 21, 2008, the entire disclosure of which is incorporated herein by reference.

The terms “top,” “bottom,” “over,” “under,” and “on” are used throughout the description with reference to the relative positions of components of the droplet actuator, such as relative positions of top and bottom substrates of the droplet actuator. It will be appreciated that the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### 6 BRIEF DESCRIPTION OF THE DRAWINGS

FIGS. 1A and 1B are illustrations showing coagulation activation on a droplet actuator.

FIGS. 2A, 2B, and 2C illustrate top views of an example of an electrode path on a droplet actuator and show a process of removing material from a blood sample using magnetically responsive beads.

FIG. 3 illustrates an embodiment of the invention in which impedance detection is used to detect a coagulated region within a droplet.

FIG. 4 shows the on-actuator standard curve for thrombin.

FIG. 5 shows on-actuator activation of thrombin generation and the resultant kinetic fluorescence curves from high, normal, and low plasma samples.

FIG. 6 shows the rate of fluorescence (from FIG. 5) fit into the standard curve to demonstrate thrombin generation curves produced on-actuator.

FIG. 7 illustrates an on-actuator methodology in accordance with an embodiment of the invention.

### 7 DESCRIPTION

The invention provides droplet actuators and methods for manipulating and testing a coagulatable sample. The invention provides techniques for causing coagulation of a coagulatable sample on a droplet actuator, conducting droplet operations using the coagulated sample or sub-components of the coagulated sample, and/or conducting testing of various components of the coagulated sample. As an example, the coagulatable sample may be a blood sample. Other coagulatable samples are described herein, e.g., see Section 7.1. The invention thus provides for the conduct of a variety of assay types making use of a coagulatable sample as an input. Examples of such assays include clot-based tests, chromogenic or color assays, direct chemical measurements, and ELISAs. As a further, non-limiting example, the invention provides a multiplexed panel of assays for assessing thrombophilia. Such a panel may, for example, include assays starting from a single blood droplet for two or more factors affecting clot formation.

### 7.1 Coagulatable Sample

Testing according to the methods of the invention requires an input sample. The input sample may be liquid that includes coagulatable components, such as coagulatable biological or non-biological components. Coagulatable components may also include artificial coagulatable substances, such as coagulatable polymers and/or beads. The coagulatable sample may be a blood sample, a milk sample, or a plant-derived sample, such as a soy milk sample. Where the coagulatable sample is a blood sample, the blood sample may include natural blood components and/or artificial blood components. Examples of blood samples include whole blood samples and samples that include various fractions of whole blood, such as samples including platelets, plasma, and/or serum. Blood samples and milk samples may be obtained from a human or non-human animal In one embodiment, the blood sample is collected from a subject. In another embodiment, the blood sample is collected from a subject undergoing mechanical circulatory support (MCS). In another embodiment, the blood sample is collected from stored blood or blood components, such as blood or blood components stored and preserved for later use in blood transfusions.

Artificial blood components may be purely artificial constructs or modified blood components, such as engineered cells and proteins. As an example, artificial blood components may include substitutes for hemostatic factors, such as artificial or modified platelets, artificial mechanical platelets or clottocytes, lyophilized platelets, infusible platelet membranes, red blood cells (RBCs) bearing RGD ligands, fibrinogen-coated albumin microcapsules, liposome-based agents, recombinant coagulation factors (e.g., Factors VII, VIII, VIIIa, and IX), recombinant activated factor VII and HLA-reduced platelets. As an example, artificial blood components may include artificial or modified oxygen carriers, red blood cell substitutes, and universal red donor cells (e.g., red blood cells in which RBC surface antigens are modified or masked, such as by binding them to a polymer, such as an mPEG polymer), stroma-free hemoglobin, modified hemoglobins (e.g., tetrameric hemoglobin, polymerized hemoglobin, conjugated hemoglobin, hemoglobin/heme vesicles, hybrid hemoglobins, recombinant hemoglobins, transgenic hemoglobins, and combinations of the foregoing), perfluorocarbon based oxygen carriers (e.g., FLUOSOL-DA®, Green Cross Corp., Japan; OXYGENT®, Alliance Corp., San Diego, Calif.). Artificial blood components may also include artificial antibodies. Other artificial or modified blood components may also be included.

The blood sample may be obtained from a subject using ordinary techniques for obtaining blood samples, e.g., using peripheral venous or arterial access or central venous or arterial access. A finger or heel stick may be used to obtain blood from a subject. For example, whole blood samples may be collected in tubes containing corn trypsin inhibitor (CTI) to inhibit contact activation. The blood sample may be one or more blood products, such as stored red blood cells, white blood cells, platelets, plasma, platelet-rich plasma (PRP), platelet-poor plasma (PPP) and/or clotting agents. Further, as noted, the sample may be obtained from an extracorporeal blood circuit, such as a circuit used for hemodialysis, hemofiltration, plasmapheresis, apheresis, and/or oxygenation. In one embodiment, the blood sample is obtained from an ECMO circuit or a cardiopulmonary bypass circuit. Blood samples may be stored for analysis and/or loaded directly onto a droplet actuator for analysis

Blood samples may be treated with one or more anticoagulants (before and/or after removal from the subject). Anticoagulated samples may be subjected to droplet operations-based protocols on the droplet actuator. Examples of suitable anticoagulants include coumarines or vitamin K antagonists (e.g., warfarin), acenocoumarol, phenprocoumon, brodifacoum, phenindione, heparin, low molecular weight heparin, synthetic pentasaccharide inhibitors of Factor Xa (e.g., fondaparinux and idraparinux), and direct thrombin inhibitors (e.g., argatroban, lepirudin, bivalirudin, and dabigatran). Anticoagulants may be used in any suitable range. Suitable concentrations of heparin may, for example, range from about 0.01 U/ml to about 1.0 U/ml. Suitable concentrations of hirudin may range from about 0.01 to about 1.5 U/ml.

A blood sample may be flowed directly from a subject into a droplet actuator reservoir. A blood sample may be flowed from a subject into a droplet actuator reservoir. A blood sample may be flowed from a subject, through a fluid path into a droplet actuator reservoir. The droplet actuator reservoir may have a vacuum established therein. The droplet actuator reservoir may include one or more substances for treating the blood, such as one or more anticoagulants. The droplet actuator reservoir may have a surface that is treated with an anticoagulant, e.g., the surface may be a heparinized surface. The blood sample may be flowed from the droplet actuator reservoir, through a fluid path or opening, into a droplet operations gap, where the droplet may be subjected to droplet operations. A blood sample may be flowed from a subject into a droplet operations gap. A blood sample may be flowed from a subject, through a fluid path into a droplet operations gap. In the droplet operations gap, the droplet may be subjected to one or more droplet operations.

The invention provides a means for testing a coagulatable sample which requires small amounts of coagulatable sample, relative to existing techniques. In one embodiment, less than about 5 mL, less than about 4 mL, less than about 3 mL, less than about 2 mL, less than about 1 mL, less than about 0.5 mL, less than about 0.1 mL, less than about 0.05 mL, less than about 0.01 mL, less than about 0.005 mL, less than about 0.001 mL, less than about 0.0005 mL, less than about 0.0001 mL, less than about 0.00001 mL, less than about 0.000001 mL, or less than about 0.0000001 mL of coagulatable sample is required as an input to the assay of the invention. In many cases, less than about 5 mL, less than about 4 mL, less than about 3 mL, less than about 2 mL, or less than about 1 mL, less than about 0.1 mL, less than about 0.05 mL, less than about 0.01 mL, less than about 0.005 mL, less than about 0.001 mL, less than about 0.0005 mL, less than about 0.0001 mL, less than about 0.00001 mL, or less than about 0.000001 mL of coagulatable sample or less than about a droplet of coagulatable sample is required for performing 2, 3, 4, 5, 6, 7, 8, 9 or 10 assays in parallel. In some cases, a sample is loaded into an on-actuator reservoir or off-actuator reservoir, and a sub-droplet for testing is dispensed into a droplet operations gap, the sub-droplet having a volume which is equal to or less than about 0.5 mL, equal to or less than about 0.1 mL, equal to or less than about 0.05 mL, equal to or less than about 0.01 mL, equal to or less than about 0.005 mL, equal to or less than about 0.001 mL, equal to or less than about 0.0001, equal to or less than about 0.00001 mL, equal to or less than about 0.000001 mL, or equal to or less than about 0.0000001 mL of coagulatable sample. Testing may be performed on the sub-droplet. Where the coagulatable sample is blood, the low volumes of sample required permit measurement or even serial measurements of coagulation in a single subject without requiring blood volumes that would result in iatrogenic anemia.

An on-actuator reservoir for receiving the coagulatable sample may be a physical reservoir and/or virtual reservoir atop an electrode in a droplet operations gap of a droplet actuator. An off-actuator reservoir may be exterior to the droplet operations gap with a fluid path coupling the off-actuator reservoir to the droplet operations gap, such that liquid flowing through the fluid path may be subjected to droplet operations in the droplet operations gap. In one embodiment, the exterior reservoir is formed in or coupled to the top substrate of the droplet actuator.

The time between sample collection and result interpretation may be significantly reduced by the invention. Reduction in time-to-result may significantly improve treatment response time. For example, prompt results can be critical in the adjustment of therapies designed to regulate coagulation. The invention thus provides a method of assessing coagulation in a subject's blood, where the assessment is accomplished in less than about 30, 25, 20, 15, 10 or 5 minutes from the time that blood is removed from the subject for testing. Further, inline testing of coagulation can be coupled to automated reporting and/or delivery of coagulation therapies in order to automate the regulation of coagulation.

A subject's coagulation system may be characterized in real time or near-real time, permitting therapy to be adjusted in real time or near-real time, e.g., during mechanical circulatory support. For example, the droplet actuator and methods of the invention are useful for managing coagulation therapy in subjects (e.g., adult or pediatric) undergoing MCS. A blood sample may be removed from the subject and/or an MCS circuit, tested, reported, and a medical care provider may adjust therapies based on the results. The droplet actuator may be provided as part of an MCS circuit, with automated sampling of blood from the circuit for testing in the droplet actuator on a periodic basis. Pamula et al., U.S. Pat. No. 7,329,545, entitled “Methods for Sampling a Liquid Flow,” granted Feb. 12, 2008, describes techniques suitable for sampling blood from a liquid flow, such as blood flow in an MCS circuit. In some embodiments, a subject's coagulation therapy may be automatically adjusted based on the results of testing.

Automated sampling may be conducted on a periodic basis. For example, a sample may be collected from a subject or from an MCS circuit at predetermined intervals. Sample sizes may be at microliter volumes or even smaller. In some embodiments, sampling may be totally automated.

Anticoagulation reagents (e.g., EDTA, sodium citrate, heparin) are typically used to prevent a blood sample from coagulating prior to analysis. For the analysis of whole blood samples where the available volume of sample is small (e.g., about 1 μl to about 20 μl), it may be undesirable to add an anticoagulation reagent directly to the sample. It may also be inconvenient to collect the blood sample into a collection device containing an anticoagulation reagent.

The invention provides a method for providing anticoagulant reagents to a small volume of blood sample to be analyzed using a droplet actuator. In this embodiment, sample wells of a droplet actuator are preloaded with anticoagulant reagents (e.g. EDTA, sodium citrate or heparin). In some cases, the anticoagulant reagents are allowed to dry in the sample wells. A small sample of whole blood (e.g., about 1 μl to about 20 μl) may be obtained, for example, by a finger stick or capillary. A drop of blood may then be placed directly into the sample well. Upon contact with the blood drop, the anticoagulant reagent will dissolve and prevent coagulation of the sample. In another embodiment, a drop of blood may be collected into a capillary tube with anticoagulants coated onto the inner walls of the capillary tube, and the capillary tube may be interfaced with the droplet actuator to input the sample.

### 7.2 Coagulating a Coagulatable Sample on a Droplet Actuator

The invention provides a method of coagulating a coagulatable sample on a droplet actuator. The coagulation may be effected in a reservoir of a droplet actuator. The reservoir may be internal or external. The coagulation may be effected in a droplet operations gap of a droplet actuator. The coagulatable sample may be partially or completely surrounded by a filler fluid when the coagulation is effected. The coagulation may be effected in a controlled manner by contacting the droplet with a procoagulant or an anticoagulant. The inventors have surprisingly discovered that droplet operations can be reliably performed on a coagulated droplet. Moreover, solid and liquid phases can be separated and subjected to further droplet operations and/or removed from the droplet actuator. For example, the solid and/or liquid phases may provide input for assays on the serum/plasma (liquid) and/or coagulated material (solid) phase.

In one aspect, the method includes providing a sample droplet including a coagulatable sample droplet on a droplet actuator and inducing or permitting coagulation in the coagulatable sample droplet to yield a coagulated droplet comprising a supernatant and coagulated material. Coagulation may be induced in an internal or external droplet actuator reservoir, and/or in a droplet operations gap of a droplet actuator. The coagulated droplet may be subjected to one or more droplet operations.

Coagulating a coagulatable sample on a droplet actuator may include contacting the coagulatable sample droplet with a procoagulant (e.g., a droplet comprising a procoagulant, a procoagulant in a filler fluid, a procoagulant on a surface) in order to induce coagulation. The procoagulant may, for example, be selected to cause, promote and/or accelerate coagulation. Examples of suitable procoagulants for blood samples include coagulation factor concentrates used to treat hemophilia, procoagulants used to reverse the effects of anticoagulants, and procoagulants to treat bleeding in patients with impaired coagulation factor synthesis or increased consumption. Additional examples include prothrombin complex concentrate, cryoprecipitate and fresh frozen plasma, Factor VII, desmopressin, tranexamic acid, aminocaproic acid, aprotinin. In certain embodiments, the coagulatable sample may include one or more coagulants, and coagulating a coagulatable sample on a droplet actuator may include incubating the sample droplet for a period of time sufficient to permit coagulation. In other embodiments, the sample droplet may be combined with a droplet comprising a procoagulant on or off the droplet actuator and incubated on the droplet actuator for a period of time sufficient to permit coagulation. Coagulation may also be induced in certain coagulatable samples by heating or cooling the sample droplet. Thus, the coagulatable sample may be incubated on a droplet actuator at a temperature selected to induce coagulation for a period of time sufficient to permit coagulation to occur.

The techniques of the invention are useful, among other things, for assessing coagulation. For example, coagulation may be assessed in the presence or absence of certain coagulants or anticoagulants. In one embodiment, timing of coagulation may be assessed. The techniques of the invention are also useful for preparing samples for analysis. A coagulated droplet may be split to yield a droplet comprising the coagulated material and a droplet substantially lacking in the coagulated material. Either droplet may be subjected to further analysis, e.g., an assay to quantify one or more substances in the droplet comprising the coagulated material and/or the droplet substantially lacking in the coagulated material.

As an example, the techniques of the invention are useful for quantifying the time course of thrombin generation following activation of the coagulation cascade. The coagulation cascade may be activated on a droplet actuator by combining a blood droplet with a droplet comprising one or more activation factors. For example, the coagulation cascade may be activated on a droplet actuator by combining a blood droplet with a droplet including a sufficient concentration of tissue factor.

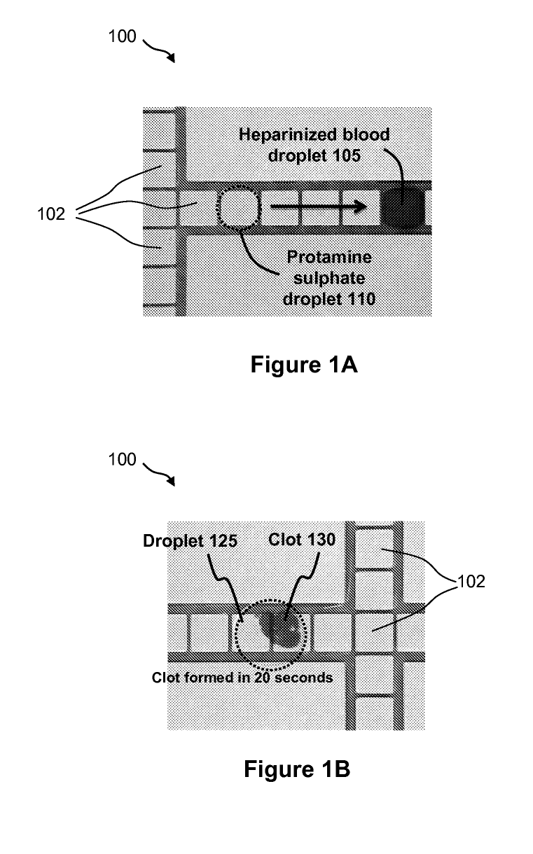

FIGS. 1A and 1B show illustrations of a process of coagulation activation on a region of a droplet actuator 100. The droplet operations are performed in a filler fluid. The filler fluid is 2 cSt silicone oil. The droplet operations are mediated by droplet transport electrodes 102. In the illustrated embodiment, the filler fluid and the droplet are sandwiched between two droplet actuator substrates in a droplet operations gap. The top substrate is a transparent cover, permitting visualization of the droplets 105, 110 and 125. A 320 nL heparinized blood droplet 105 is provided on the surface of droplet actuator 100 in the filler fluid, dispensed from a droplet actuator reservoir (not shown). A 320 nL protamine sulphate droplet 110 is also provided on the surface of droplet actuator 100 in the filler fluid, dispensed from a droplet actuator reservoir (not shown). Droplet operations are used to combine heparinized blood droplet 105 with protamine sulphate droplet 110 in order to provide a combined droplet 125 in which the anticoagulant effects of heparin are at least partially inhibited or neutralized, thereby permitting activation of the coagulation pathway. Following activation of the coagulation pathway, discrete solid and liquid phase components (i.e., coagulated material 130) can be distinguished within a droplet of blood, as shown in FIG. 1B. The coagulated droplet remains subject to droplet operations.

The filler fluid is immiscible with the coagulatable droplet. The filler fluid may be a liquid filler fluid that is immiscible with the coagulatable droplet. The filler fluid may be an oil, such as a silicone oil, an alkane oil, and/or a fluorinated oil. The oil may be doped with a surfactant, e.g., Span 85. Other examples of filler fluid formulations suitable for use in the invention may be found in U.S. patent application Ser. Nos. 11/639,594, entitled “Filler Fluids for Droplet Operations,” filed on Dec. 15, 2006; 61/141,083, entitled “Enhancing and/or Maintaining Oil Film Stability in a Droplet Actuator,” filed on Dec. 29, 2008; 61/092,278, entitled “Droplet actuators, Modified Fluids and Methods,” filed on Aug. 27, 2008; 61/094,891, entitled “Droplet Actuators, Modified Fluids and Methods,” filed on Sep. 6, 2008; 61/140,703, entitled “Oil Film Stability on a Droplet Actuator,” filed on Dec. 24, 2008; and International Patent Application No. PCT/US2008/072604, entitled “Use of Additives for Enhancing Droplet Actuation,” filed on Aug. 8, 2008; the entire disclosures of the foregoing patent applications and their priority documents are incorporated herein by reference for their teaching concerning filler fluid formulations.

It should also be noted that a coagulated droplet may be dissolved on a droplet actuator. For example, droplet comprising coagulated blood may be contacted with a thrombolysis agent, such as a clot-degrading enzyme, a plasma activator agent, and/or a plasminogen activator agent. Examples of suitable clot-degrading enzymes include tenzymes that degrade fibrin strands within the clot. Examples of suitable plasma activator agents, include agents which increase plasma activator activity. Examples of suitable plasminogen activators, include streptokinase, urokinase, and tissue plasminogen. The droplet comprising coagulated blood may be contacted with a thrombolysis agent by combining the coagulated blood droplet with a droplet comprising a thrombolysis agent. The droplet comprising coagulated blood may be contacted with a thrombolysis agent by providing the coagulated blood droplet in a filler fluid comprising a thrombolysis agent. The droplet comprising coagulated blood may be contacted with a thrombolysis agent by adding a thrombolysis agent to the coagulated blood droplet and/or transporting the droplet comprising coagulated blood into contact with a thrombolysis agent.

### 7.3 Manipulating a Coagulated Sample on a Droplet Actuator

The inventors have surprisingly discovered that coagulated sample can be manipulated on a droplet actuator. The invention provides a method of conducting droplet operations on a droplet that contains coagulated sample. The method may involve providing the coagulated sample droplet on the droplet actuator and subjecting the coagulated sample droplet to droplet operations. The droplet operations may be electrode-mediated, e.g., electrowetting mediated or dielectrophoresis mediated. For example, in one embodiment the droplet operation is selected from the group consisting of: dispensing one or more droplets from a coagulated sample droplet; splitting, separating or dividing a coagulated sample droplet into two or more droplets; transporting a coagulated sample droplet from one location to another in any direction; merging or combining two or more droplets including at least one coagulated sample droplet into a single droplet; diluting a coagulated sample droplet; mixing a coagulated sample droplet; agitating a coagulated sample droplet; deforming a coagulated sample droplet; retaining a coagulated sample droplet in position; incubating a coagulated sample droplet; heating a coagulated sample droplet; cooling a coagulated sample droplet; disposing of a coagulated sample droplet; transporting a coagulated sample droplet out of a droplet actuator; and/or any combination of the foregoing.

Coagulated blood can be manipulated on a droplet actuator. Thus, in one embodiment, the invention provides a method of conducting droplet operations on a droplet that contains coagulated blood. The method may involve providing the coagulated blood droplet in a droplet operations gap of a droplet actuator and subjecting the coagulated blood droplet to droplet operations. The droplet operations may be electrode-mediated, e.g., electrowetting mediated or dielectrophoresis mediated. For example, in one embodiment the droplet operation is selected from the group consisting of: dispensing one or more droplets from a source coagulated blood droplet; splitting, separating or dividing a coagulated blood droplet into two or more droplets; transporting a coagulated blood droplet from one location to another in any direction; merging or combining two or more droplets including at least one coagulated blood droplet into a single droplet; diluting a coagulated blood droplet; mixing a coagulated blood droplet; agitating a coagulated blood droplet; deforming a coagulated blood droplet; retaining a coagulated blood droplet in position; incubating a coagulated blood droplet; heating a coagulated blood droplet; cooling a coagulated blood droplet; disposing of a coagulated blood droplet; transporting a coagulated blood droplet out of a droplet actuator; and/or any combination of the foregoing.

The foregoing droplet operations may be effected on a coagulated sample droplet and/or coagulated blood droplet which is partially or substantially completely or completely bounded by a filler fluid. The filler fluid may, for example, include a liquid filler fluid. The liquid filler fluid may, for example, include an oil filler fluid. The oil filler fluid may, for example, include a silicone oil, an alkane oil, and/or a fluorinated oil.

### 7.4 Separating Blood Components

In certain embodiments of the invention, it may be useful to separate blood components on a droplet actuator. For example, a coagulatable droplet may be coagulated on a droplet actuator, and the coagulated components may be separated from the supernatant or uncoagulated components. The separation may be effected on a coagulated sample droplet and/or coagulated blood droplet which is partially or substantially completely or completely bounded by a liquid filler fluid. The separation may yield one or more daughter droplets including supernatant and substantially lacking in coagulated material. The separation may yield one or more daughter droplets including the coagulatable material with a reduced amount of the supernatant. In some cases, the coagulatable material may be washed to substantially completely remove the supernatant. A coagulated droplet may be split to yield a droplet comprising the coagulated material and a droplet substantially lacking in the coagulated material. Droplets produced by the process may be subjected to further analysis, e.g., an assay to quantify one or more substances in the droplet comprising the coagulated material and/or the droplet substantially lacking in the coagulated material.

In one aspect of the invention, the method includes providing a sample droplet including a coagulatable sample droplet on a droplet actuator, inducing or permitting coagulation in the coagulatable sample droplet to yield a coagulated droplet comprising a supernatant and coagulated material, and separating one or more components of the coagulated droplet. For example, the coagulated droplet may be subjected to an electrode-mediated droplet splitting operation to yield a droplet comprising the coagulated material and a droplet substantially lacking in the coagulated material. As another example, one or more droplets of supernatant may be dispensed from the coagulated droplet.

In another aspect of the invention, magnetically responsive beads may be provided in a coagulatable droplet. Upon coagulation, the magnetically responsive beads may be trapped in the coagulated portion(s) of the coagulated droplet. A magnetic field may be used to immobilize the coagulated portion(s) of the coagulated droplet in order to execute a washing protocol to remove the uncoagulated portion of the coagulated droplet. The uncoagulated material with magnetically responsive beads may, for example, be subjected to a merge-and-split washing protocol, yielding a droplet including the coagulated material and substantially lacking in supernatant from the coagulated droplet. The coagulated material may be dissolved and subjected to further analysis.

Similarly, a droplet-based washing protocol may be mediated without using magnetically responsive beads by using a physical barrier to restrain the coagulated material. A physical barrier may be used to permit removal of some or all of the liquid volume of the droplet surrounding the coagulated material. The physical obstacle may, for example, include a membrane, sieve, and/or projection from the droplet actuator (e.g., from the top plate and/or bottom plate). Where a physical obstacle (projection or object) attached to the top plate and/or bottom plate is employed, it should be arranged so as to permit droplet transport on the droplet operations surface mediated by droplet operations electrodes, while preventing the coagulated material from following, e.g., using a projection from the top plate that leaves sufficient space for droplet transport and/or a projection with one or more openings that permits the droplet to be transported through the opening while trapping the coagulated material. In some embodiments, the physical barrier may be coated with anticoagulants or procoagulants so that when a sample droplet contacts the physical barrier anticoagulation or procoagulation is initiated, enhanced or modulated. In the case of procoagulation, the physical barrier may thus serve the dual purpose of providing the procoagulant into the droplet, and also restraining the coagulated portion of the sample droplet.

In other embodiments, it may be useful to remove one or more substances from a coagulatable droplet without relying on coagulation. For example, in some embodiments, it may be useful to remove red blood cells or hemoglobin in a targeted manner. For the analysis of whole blood samples where the available volume of sample is small (e.g., about 1 μl to about 20 μl), it may be difficult to obtain adequate sample free of red blood cells (RBC) or free hemoglobin. Contaminating RBC and/or free hemoglobin may cause assay interference and/or disruption in detection when using, for example, an optical based assay. Standard methods (e.g., filtration) typically used to remove materials such as RBC and/or hemoglobin are difficult to perform on a small sample of blood and may fail to provide sufficient filtrate for subsequent analysis. The invention provides methods of removing material (e.g., RBC, hemoglobin) from a small volume of blood sample using a droplet actuator.

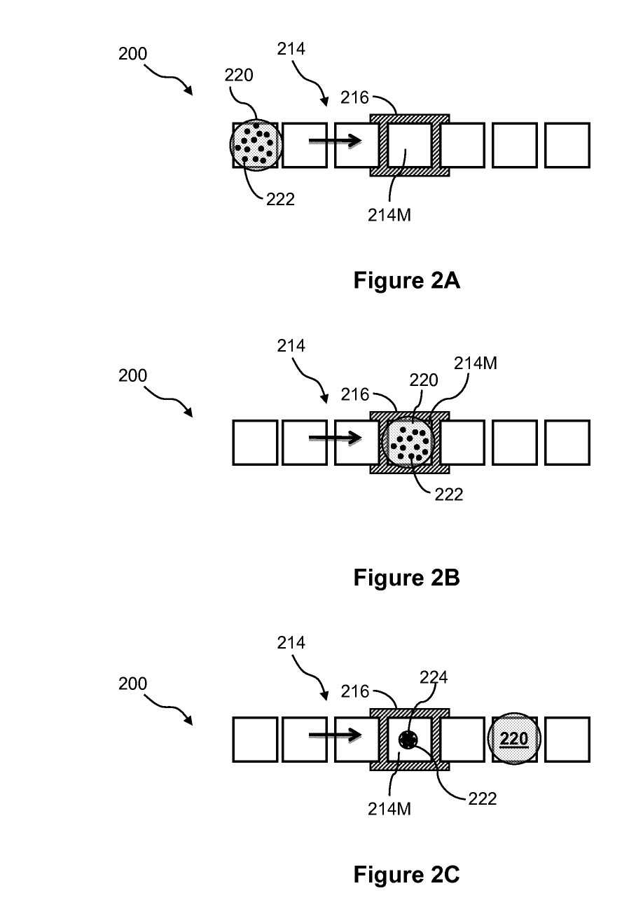

FIGS. 2A, 2B, and 2C illustrate top views of a region of a droplet actuator 200 and show an illustrative process for removing material from a blood sample using magnetically responsive beads. In one embodiment, the method of the invention is used to remove red blood cells from a blood sample prior to analysis. In an alternative embodiment, the method of the invention is used to remove hemoglobin from a blood sample that contains lysed red blood cells prior to analysis.

Droplet actuator 200 may include a path or array of droplet operations electrodes 214 (e.g., configured for electrowetting and/or dielectrophoresis). Droplet operations electrodes 214 may be configured for conducting one or more droplet operations on a droplet operations surface of the droplet actuator. In some cases, the droplet operations surface may be provided within a droplet operations gap of the droplet actuator. A magnet 216 is arranged in proximity to droplet operations electrodes 214. As illustrated, magnet 216 is arranged such that one or more of the droplet operations electrodes (e.g., droplet operations electrode 214M) is/are within the magnetic field of magnet 216, or similarly, magnet 216 is arranged such that a droplet path established by the electrodes is within the magnetic field of magnet 216. Magnet 216 may be a permanent magnet or an electromagnet or any other magnetic field emitting device. Droplet actuator 200 may include a bead-containing droplet 220 on the droplet operations surface. Bead-containing droplet 220 may include one or more beads 222. Beads 222 may have an affinity for one or more components of the coagulatable sample, such as an affinity for red blood cells (e.g., beads 222 include anti-RBC antibodies). In another example, beads 222 may have an affinity for hemoglobin (e.g., beads 222 include anti-hemoglobin antibodies). Droplet operations electrodes 214 may be used to mediate various droplet operations using bead-containing droplet 220 on the droplet operations surface overlying droplet operations electrodes 214. For example, droplet 220 may be transported along the path of, or any path established by, droplet operations electrodes 214.

FIG. 2A shows a step in a process of removing material (e.g., RBC or hemoglobin) from a blood sample. In this step, bead-containing sample droplet 220 is provided on the droplet operations surface. Bead-containing sample droplet 220 may, for example, include a few microliters (e.g., about 1 μl to about 20 μl) of blood and beads 222 having affinity for the material that is to be removed. Bead-containing sample droplet 220 may be provided by mixing beads 222 and sample in a sample reservoir. In some cases, mixing of beads and sample in the sample reservoir may be enhanced using a sonicator. Beads and sample may be incubated in the reservoir for a period of time sufficient to permit binding of the target material to beads 222. In various alternatives, the sample may be loaded into a reservoir that already includes a bead-containing droplet or the beads and/or bead-containing droplet may be loaded into a reservoir that already includes the sample. In another alternative, a sample droplet and a bead-containing droplet may be combined on the droplet actuator using droplet operations affected by droplet operations electrodes. FIG. 2B shows another step of the process, in which bead-containing droplet 220 is transported via electrode-mediated droplet operations away from a sample reservoir and to droplet operations electrode 214M. FIG. 2C shows a third step in which bead-containing droplet 220 is transported away from droplet operations electrode 214M along a path of droplet operations electrodes 214. As bead-containing droplet 220 moves away from droplet operations electrode 214M, beads 222 remain trapped in the magnetic field in a concentrated bead droplet 224. Bead droplet 224 is retained by magnet 216.

By use of the steps shown in FIGS. 2A, 2B, and 2C, beads 222 that include bound RBC, hemoglobin or another target substance are separated from the original bead-containing droplet 220 to form a substantially bead-free (and target-substance-free) droplet 220 (e.g., serum or plasma). Concentrated bead droplet 224 may, for example, be discarded (e.g., transported using droplet operations to a waste reservoir; not shown) or subjected to further droplet operations, e.g., as part of another assay. For example, a buffer droplet may be transported onto electrode 214M to merge with the trapped bead-containing droplet. The merged droplet may be used to conduct one or more steps in an assay protocol.

Beads with anti-hemoglobin antibodies may be used to capture and remove hemoglobin from a blood sample. In this example, a lysis agent (e.g., a detergent or hypotonic buffer) may be added to the sample droplet to lyse red blood cells. Free hemoglobin binds to beads and may be removed from bead-containing droplet using droplet operations as described above or other droplet wash protocols.

In an alternative embodiment the magnetically responsive beads are replaced with beads which are not substantially magnetically responsive. The magnet may be replaced with one or more physical barriers as a means for immobilizing the beads. For example, the droplet actuator may include: a base substrate comprising electrodes configured for conducting droplet operations on a droplet operations surface thereof a droplet comprising one or more beads situated on the droplet operations surface; a barrier arranged in relation to the droplet and the electrodes such that a droplet may be transported away from the beads using one or more droplet operations mediated by one or more of the electrodes while transport of the beads is restrained by a barrier. In this manner, a droplet is produced substantially lacking in the beads. Where the beads are bound to RBCs or other target components of the droplet, the bound RBCs and other target components are removed from the droplet.

In some cases, the droplet actuator also includes a top substrate, such as a top substrate, separated from the droplet operations surface to form a gap for conducting droplet operations. When a top substrate is present, the barrier may be mounted on the top substrate and may extend downward from the top substrate. The barrier may be configured to leave a gap between a bottom edge of the barrier and the droplet operations surface. A droplet may be transported through the gap while the barrier restrains transport of the beads. In this manner, a droplet is produced substantially lacking in the beads.

In some embodiments, the barrier may include a vertical gap through which fluid may pass during a droplet operation mediated by one or more of the electrodes. When present, the vertical gap may, in certain embodiments, be situated over an electrode. In some embodiments, the vertical gap extends substantially from a surface of the top substrate facing the gap and the droplet operations surface. A droplet may be transported through the vertical gap while the barrier restrains transport of the beads. In this manner, a droplet is produced substantially lacking in the beads.

In some embodiments, the droplet actuator of the invention includes one or more beads completely surrounded by and/or trapped the barrier. In such an embodiment, the one or more beads are blocked by the barrier from being transported away from the barrier enclosure in any direction, while permitting droplets to be transported into and out of the barrier's enclosure. For example, the barrier may extend from the top substrate and leave a gap between a bottom of the barrier and the bottom substrate. The barrier may be an enclosed barrier of any shape situated on a path of electrodes configured for transporting droplets into contact with and away from beads which are trapped within the confines of the barrier. The droplets may, for example, contain reagents, samples, and/or smaller beads which are sufficiently small to be transported into and out of the barrier.

In other embodiments, the barrier may include an angular barrier traversing an electrode path and pointing in a direction which is away from a bead retaining area of the barrier. In a similar embodiment, the barrier may include an angular barrier traversing an electrode path and pointing in a direction which is towards a bead retaining region of the barrier. A droplet may be transported out of the barrier enclosure while the barrier restrains transport of the beads. In this manner, a droplet is produced substantially lacking in the restrained beads.

International Patent Application No. PCT/US08/74151, entitled “Bead Manipulations on a Droplet Actuator,” filed on Aug. 25, 2008, includes various physical barrier arrangements for washing beads; the entire disclosure is incorporated herein for its teaching concerning restraining beads during droplet operations.

In another embodiment that does not make use of beads, the surface of the droplet actuator may be coated with materials that will deplete the droplet of hemoglobin or red blood cells or other matter. A blood droplet can be transported over a zone on the droplet actuator with anti-RBC antibodies. By incubating the droplet or transporting the droplet a certain number of times over that zone, all the RBCs can be depleted from the droplet.

In various embodiments, the method yields a droplet which is substantially free of beads. In other embodiments, the method yields a droplet which is substantially free of beads which are restrained by the physical barrier or magnet, i.e., other beads not so restrained may remain in the droplet. For example, magnetically responsive beads may be removed, while beads that are not substantially magnetically responsive may remain in the droplet. Similarly, beads large enough to be restrained by a physical barrier may be removed, while beads which are too small to be blocked by the physical barrier may remain in the droplet. In various embodiments, the method yields a droplet in which at least 90%, 95%, 99%, 99.9%, 99.99%, 99.999%, or 99.9999% of beads are removed from the starting bead-containing droplet. In other embodiments, the method yields a droplet in which at least 90%, 95%, 99%, 99.9%, 99.99%, 99.999%, or 99.9999% of magnetically responsive beads are removed by a magnetic field from the starting bead-containing droplet. In other embodiments, the method yields a droplet in which at least 90%, 95%, 99%, 99.9%, 99.99%, 99.999%, or 99.9999% of beads are removed by a physical barrier from the starting bead-containing droplet.

In another embodiment, the methods are applied to remove target components from a droplet. The target components may, for example, be cells, such as plant, animal, protozoan or fungal cells; tissues; multicellular organisms; organelles; and chemical compounds. In some cases, method yields a droplet in which at least 90%, 95%, 99%, 99.9%, 99.99%, 99.999%, or 99.9999% of the target component is removed from the starting bead-containing droplet.

The invention also provides a droplet actuator comprising or associated with a magnet of sufficient strength to restrain magnetic beads from further transport when a droplet comprising magnetic beads is transported using droplet operations on a droplet operations surface into proximity with the magnet. The invention also provides a droplet actuator comprising or associated with a magnet of sufficient strength to snap a sub-droplet including beads from a droplet including magnetic beads is transported using droplet operations on a droplet operations surface into proximity with the magnet. In one embodiment, the concentration of the beads can be chosen to be very high such that when a blood droplet combined with anti-RBC magnetic beads is moved into a magnetic field, all the beads are attracted towards the magnet and are pulled out of the bulk of the sample droplet, thereby depleting substantially all RBCs and/or hemoglobin along with the beads from the sample.

In yet another embodiment, one or more products of the process of separating blood components are removed from the droplet actuator. For example, coagulated material, uncoagulated material, blood lacking RBCs, blood lacking free hemoglobin, etc., may be removed from the droplet actuator for further processing. In one embodiment, supernatant from a coagulated droplet is transported into proximity with an opening extending from the droplet operations gap of a droplet actuator to an exterior of the droplet actuator. The supernatant may be removed from the droplet operations gap via the opening and subjected to further analysis. In one embodiment, the opening includes a capillary, and the supernatant enters the capillary as a result of capillary forces.

### 7.5 Assaying a Coagulatable Sample

The invention provides techniques for assaying coagulatable samples. The assay or assays may be directed towards an understanding of the coagulation process itself, such as diagnosis of a coagulation disorder, or testing the affect of a therapeutic agent on coagulation. In other embodiments, the coagulation may be viewed as a sample preparation step and the assay or assays may be directed towards identifying and/or quantifying a component of a coagulated portion of a coagulated droplet and/or supernatant produced as a result of coagulation. With respect to assays directed towards an understanding of the coagulation process itself, in some embodiments, the assay techniques involve assessment as components of a droplet are in the process of coagulating. For example, one or more coagulation factors may be assayed at various stages of coagulation.

In medical applications, the invention provides for coagulability testing in subjects. In some cases, coagulability may be routinely monitored. Routine monitoring is critical for decision-making in cardiovascular medicine and surgery. Most of the morbidity and mortality associated with cardiovascular disorders relate to complications of bleeding or thrombosis.

Regulation of the hemostatic system has become an important adjunct to the treatment of cardiovascular disorders. Coagulability testing may, for example, be used to monitor coagulability changes resulting from the use of therapies, such as anticoagulants, procoagulants, MCS, and/or artificial blood components. Coagulability testing may be useful for assessing any change in coagulability, such as changes leading to hypocoagulability or hypercoagulability.

The invention provides a medical monitoring device that includes a sampling line coupled in fluid communication with a blood source. The blood source may, for example, be a heparinized catheter or an in-line access point on an extracorporeal circulation device (such as an extracorporeal membrane oxygenation device or a circulation assist device). The sampling line is configured to flow blood to a droplet actuator for processing. For techniques for sampling droplets from a continuous liquid flow, see Pamula et al., U.S. Pat. No. 7,329,545, entitled “Methods for Sampling a Liquid Flow,” granted on Feb. 12, 2008, the entire disclosure of which is incorporated herein by reference. The device may be scheduled to sample a small volume of blood at routine intervals and/or the sampling may be triggered by other parameters being monitored by the system. Sampling may include using the droplet actuator to dispense one or more sub-droplets of sample for testing. Various assays may be performed using the sub-droplets. Examples of suitable assays are described herein, and the assays described herein and in International Patent Application No. PCT/US2006/47486, entitled “Droplet-based biochemistry,” filed on Dec. 11, 2006, the entire disclosure of which is incorporated herein by reference.

The invention provides assays for the assessment of coagulation. For example, the invention provides assays for assessing various elements of coagulation cascades, such as the blood clotting cascade. Examples include immunoassays relating to Factor V Leiden, Factor V, Factor Va, Factor VII, Factor VIIa, Factor IX, Factor IXa, Factor X, Factor Xa, Factor XI, Factor XIa, Factor XII, Factor XIIa, Factor XIII, Factor XIIIa, fibrin, cross-linked fibrin, fibrin degradation products, fibrinogen, homocysteine, kallikrein, kininogen, plasmin, plasminogen, prekallikrein, prothrombin, prothrombin degradation products, prothrombin fragment 1+2, Protein C, Protein S, thrombin, thrombin complexes, thrombin-antithrombin complexes, antithrombin, tissue factor, tissue plasminogen activator, and anti-cardiolipin antibody. Various assay steps of the assays may be performed on a droplet actuator using droplet operations. A combination of any of the foregoing assays or any of the foregoing assays with other assays may be provided on a single droplet actuator. Testing may proceed for any of the foregoing assays or any of the foregoing assays with other assays using a single droplet of blood. In certain embodiments, the sample droplet is divided using droplet operations into multiple subsample droplets, and each subsample droplet is used in an assay protocol for a single analyte. This sample multiplexing approach avoids the problem of cross-reactivity between antibodies. In another example, multiple analytes are analyzed in each subsample, but antibodies susceptible to cross-reactivity problems are included in separate subsample droplets.

The invention provides assays for the assessment of thrombin generation. In some cases, the assays are useful for assessing generation of thrombin over time. The assays may make use of surrogate markers for thrombin generation, such as prothrombin fragments, thrombin complexes, and other markers of thrombin production or activity. An example of a suitable prothrombin fragment is prothrombin fragment 1+2 (F1+2). An example of a suitable thrombin complex is thrombin-antithrombin complex (TAT).

The assays may be useful for assessing the time course of thrombin generation following activation of the clotting cascade. The assays may be useful for assessing thrombin generation following activation using various amounts of coagulation agent. The assays may be useful for assessing thrombin generation in samples with varying concentrations of anticoagulation agents.

The assays may be useful for assessing coagulation in a subject. The subject may be a human or non-human animal Diagnostic information from the assays may be correlated with various clinical conditions. For example, concentrations of TAT and F1+2 are elevated in patients with peripheral artery disease, and F1+2 is elevated in acute thrombotic conditions such as myocardial infarction. As another example, thrombophilia is associated with protein C, protein S, or antithrombin III deficiency, elevated Factor VIII or homocysteine levels, and presence of anti-phospholipid antibody syndrome. The presence of Factor V Leiden and prothrombin 20210A mutations are associated with a hypercoagulable state. Diagnostic information from assays of the invention may be correlated with various coagulation disorders, such as hemophilias and thrombophilias. Diagnostic information from assays of the invention may also be useful for monitoring and managing procoagulation and anti-coagulation therapies.

Samples used in the assays may include blood samples. Blood samples may, for example, be as described in Section 7.1. As noted there, the input blood sample may, among other things, include whole blood or plasma. In various embodiments, the input blood sample may consist substantially of whole blood or may consist substantially of plasma. Where plasma is used as the input blood sample, it may, for example, be PRP or PPP.

As an example, an assay may be executed beginning with a whole blood sample. The whole blood sample may be loaded on the droplet operations gap of a droplet actuator and/or into a reservoir for loading onto the droplet actuator. Droplet operations may be used for dispensing and distributing one or more sub-droplets from the whole blood sample to various regions of the droplet actuator. The whole blood sample may be subjected to coagulation, manipulation, and/or separation steps, such as those described herein. The separated blood components may be used as inputs for the assays of the invention. Measurement of TAT and F1+2 concentrations, for example, may be conducted using the supernatant created by effecting coagulation in a blood droplet on a droplet actuator.

A starting sample may be divided into sub-samples, and the sub-samples may be subjected to a variety of assay protocols on one or more droplet actuators. A sample may be loaded on a droplet actuator, divided or dispensed using droplet operations into sub-samples. The sub-samples may be subjected to a variety of coagulation, manipulation, separation steps, and assay protocols on the droplet actuator. A blood sample may be loaded on a droplet actuator, divided into sub-samples, and some or all of the sub-samples may serve as multiplicates, subjected to coagulation, manipulation, separation steps, and assay protocols on the droplet actuator. Multiple samples (e.g., different subjects, different collection points on the same subject, and/or different collection times on the same subject) may be subjected to a variety of coagulation steps, manipulation steps, separation steps, and assay protocol steps on the droplet actuator. Multiple samples may be divided into sub-samples, and the sub-samples may be subjected to a variety of coagulation steps, manipulation steps, separation steps, and assay protocol steps, where one or more subgroup of the steps is effected on a first droplet actuator and one or more subgroups of the steps is effected on a second droplet actuator or without use of a droplet actuator. Multiple samples may be loaded on a droplet actuator and subjected to a variety of coagulation steps, manipulation steps, separation steps, and assay protocol steps on the droplet actuator. Multiple samples may be loaded on a droplet actuator, divided into sub-samples, and the sub-samples may be subjected to a variety of coagulation steps, manipulation steps, separation steps, and assay protocol steps on the droplet actuator. Multiple samples may be loaded on a droplet actuator, divided into sub-samples, and some or all of the sub-samples may serve as multiplicates, subjected to a common coagulation steps, manipulation steps, separation steps, and assay protocol steps on the droplet actuator.

A subset of the droplet operations of an assay may be synchronized for different assay protocols or multiplicates of the same protocol. For example, one or more of the following operations may be synchronized: coagulation activation (e.g., simultaneous mixing of tissue factor with sample), quenching (e.g., sequential transportation and mixing of quenching solution with activated sample), bead washing, luminescence detection, and fluorescence detection.

Thrombin generation may be assessed upon activation of the clotting cascade. The clotting cascade may be activated using a coagulation agent, such as human tissue factor. A droplet of blood may be combined using droplet operations with a droplet comprising tissue factor to initiate coagulation. The droplet of blood and the droplet comprising tissue factor may be combined on a droplet operations surface of a droplet actuator. The droplet of blood and the droplet comprising tissue factor may be combined in a droplet operations gap of a droplet actuator. The droplet of blood, the droplet comprising tissue factor, and the resulting coagulating droplet, may be partially or substantially completely or completely bounded by a liquid filler fluid, such as a filler fluid consisting essentially of an oil, such as a silicone oil, an alkane oil, and/or a fluorinated oil.

The assay of the invention may include the use of droplet operations to combine a droplet of plasma with a droplet comprising a known concentration of thrombin, and measuring the time to clot formation in the combined droplet. The plasma may, for example, be PRP or PPP. The droplet of plasma, droplet comprising a known concentration of thrombin, and/or the combined droplet may be partially or substantially completely or completely bounded by a liquid filler fluid during the droplet operations and/or detection of the result. The filler fluid may in some cases consist essentially of an oil, such as a silicone oil, an alkane oil, and/or a fluorinated oil. PPP and PRP may be obtained, for example, by centrifugation of whole blood. The PPP or PRP may be loaded into a droplet actuator reservoir and/or into a droplet operation gap, and divided or dispensed using droplet operations into sub-droplets suitable for conducting the assay.

In embodiments in which the formation of coagulated material may interfere with the measurements from the droplet, solid and liquid phases may be separated using the techniques described herein. The clotted portion of the droplet may be removed using magnetically responsive beads having affinity to the clotted portion. The magnetically responsive beads may be immobilized using a magnetic field, and the plasma may be transported away from the immobilized beads using droplet operations. The solid coagulated material is left behind, and assays, such as assays, may be performed on the resultant plasma. The remaining coagulated material may be subjected to additional droplet operations based protocols for further analysis.

Alternatively, the coagulated material incorporating magnetically responsive beads may be pulled aside within an elongated droplet so that no beads are exposed to the detection window during detection. The magnet may, of course, be provided in a variety of arrangements in relation to the droplet operations surface or droplet operations. For example, the magnet may be situated under the droplet operations surface, atop the droplet actuator, laterally adjacent to the droplet actuator, in the droplet actuator gap, and/or in or partially in one or more of the substrates forming the droplet actuator. In short, the magnet may be provided in any position which attracts the beads to a region of the droplet which is outside of or at least substantially outside of the detection window. In an alternative embodiment, the magnet may pull the beads entirely out of the droplet that is being subjected to detection. For example, a droplet actuator may include a powerful magnet in a region of the droplet actuator established for bead removal. The power of the magnet may be selected to pull magnetic beads out of any droplet which is moved into the bead removal region of the droplet actuator. In some cases, removal of the beads may effectively be irreversible.

To elaborate further, the invention provides a method of detecting an analyte. The method may include providing in a detection window a droplet. The droplet may include a signal-producing substance indicative of the presence and/or quantity of an analyte. The droplet may include one or more magnetically responsive beads bound to a coagulated material which may interfere with signal produced by the signal producing substance. The method may include using a magnetic field for magnetically removing the magnetically responsive beads and bound coagulated material from the detection window, and/or magnetically restraining the magnetically responsive beads and bound coagulated material from entering the detection window while transporting and/or retaining the droplet in the detection window. The transporting and/or retaining the droplet in the detection window may be electrode-mediated.

The method may include using physical barrier for restraining the coagulated material from entering the detection window while transporting and/or retaining the droplet in the detection window. The transporting of the droplet into and/or retaining of the droplet in the detection window may, for example, be electrode mediated. It will be appreciated that this physical barrier approach may be used regardless of whether or not beads are included in or bound to the coagulated material.

The method may include detecting a signal produced by the signal-producing substance without substantial interference from the magnetically responsive beads and/or coagulated material. The invention provides a method of detecting an analyte including providing in a detection window a droplet, where the droplet includes a signal-producing substance indicative of the presence and/or quantity of an analyte and a coagulated material, which coagulated material may interfere with signal produced by the signal producing substance.

In the method of detecting an analyte, the droplet may be provided in a droplet operations gap of a droplet actuator. The detection window may include an actual opening or window in a substrate of the droplet actuator. The detection window may include a region of sensitivity for detection of signal by a sensor. With respect to the embodiment making use of magnetically responsive beads, using a magnetic field may include providing a fixed magnet in proximity to the detection window. Transporting the droplet into the detection window may deliver the magnetically responsive beads and coagulated material into sufficient proximity with the fixed magnet that the beads may be pulled away from and/or restrained from entering the detection window. With respect to the embodiment making use of physical barrier, transporting the droplet into the detection window may be accomplished while the coagulated material is restrained from progressing into the detection window by a physical barrier. This restraining of coagulated material from entering the detection window may be accomplished with or without removing the coagulated material from the droplet.

In these and any other embodiments of the invention making use of a magnetic field, the magnetic field may be generated by any suitable magnetic field source. For example, the magnetic field source may include a fixed permanent magnet, a moveable permanent magnet, and/or an electromagnet. The magnetic field may be arranged to aggregate the magnetically responsive beads at an edge of the droplet. The magnetic field may be arranged to aggregate the magnetically responsive beads with the coagulated material in a region of the droplet which may be outside the detection window or outside the region of the droplet being subjected to detection. In some cases, the magnetic field is selected to break the magnetically responsive beads away from the droplet. For example, the magnetic field may break the magnetically responsive beads with coagulated material away from the droplet while the droplet may be being held in place and/or moved by electrode mediated forces. In some cases, the magnetic field attracts the magnetically responsive beads with coagulated material in a manner which pulls them with the coagulated material to an edge of the droplet while the droplet may be at least partially in the detection window. In some cases, the magnetic field pulls the magnetically responsive beads with coagulated material out of the droplet as the droplet passes over the magnet. In some cases, the magnetic field pulls the magnetically responsive beads with coagulated material out of the droplet as the droplet approaches a vicinity of the magnet. In some cases, the magnetic field pulls the magnetically responsive beads with coagulated material out of the droplet as the droplet approaches the detection window. In some cases, the magnetic field attracts the magnetically responsive beads with coagulated material in a manner which restricts substantially all of the beads from entering or re-entering the detection window as the droplet may be transported into the detection window.

The droplet actuator may, for example, include a plurality of paths of electrodes associated with the droplet operations substrate, each path associated with a detection window, and a magnetic field in proximity to the path arranged for magnetically removing the magnetically responsive beads and coagulated material from the corresponding detection window, and/or magnetically restraining the magnetically responsive beads with coagulated material from entering the corresponding detection window while transporting into and/or retaining the droplet in the detection window. The droplet may emit a signal indicative of the presence, absence and/or quantity of one or more analytes.

In one embodiment, the invention provides simultaneous assay on a single droplet actuator using droplet operations protocols for both ELISA and functional (enzymatic cleavage) assays. The quantity of analyte may be determined by measuring the fluorescence or color or luminescence or electrochemical signal or other enzymatically produced signal from a droplet on a droplet actuator, or any combination of the foregoing signal types or the foregoing signal types with other signal types. In one embodiment, the fluorescence is generated by cleavage of the fluorogenic substrate Z-Gly-Gly-Arg-amino-methyl-coumarin (Z-AMC) in a droplet on a droplet actuator. The droplet may in some cases be partially or substantially completely or completely bounded by a liquid filler fluid during detection. For example, the filler fluid may consist essentially of an oil, such as a silicone oil, an alkane oil, and/or a fluorinated oil.

The invention provides a droplet-based ELISA for TAT complexes and/or F1+2. The ELISA may, for example, be performed on the droplet actuator using a bead substrate. Focusing on the F1+2 ELISA, beads may be provided having affinity for F1+2. The beads may, for example, be coated with or otherwise bound to antibody and/or antibody fragments specifically binding to F1+2. If necessary, components such as thrombin to which the antibody and/or antibody fragments also bind may be removed from the sample prior to initiation of the assay. Bead-containing droplets may be positioned in a droplet operations gap, and each bead-containing droplet may be combined using droplet operations with standard droplet and/or a sample droplet. Alternatively, F1+2 beads may be combined with a sample droplet in a droplet actuator reservoir. Any F1+2 present is bound to the beads. A droplet including an enzyme-linked antibody specific for F1+2 may be added to the existing droplet reaction. Following execution of a bead washing protocol to remove unbound antibody-enzyme reagent, a droplet comprising a substrate solution may be added to the droplet reaction, causing a signal (e.g., color, fluorescence or luminescence) which is proportional to the amount of captured F1+2. The signal may be measured using an appropriate sensor. A similar protocol may be utilized for other elements of the coagulation cascade or related processes, such as Factor V Leiden, Factor V, Factor Va, Factor VII, Factor VIIa, Factor IX, Factor IXa, Factor X, Factor Xa, Factor XI, Factor XIa, Factor XII, Factor XIIa, Factor XIII, Factor XIIIa, fibrin, cross-linked fibrin, fibrin degradation products, fibrinogen, homocysteine, kallikrein, kininogen, plasmin, plasminogen, prekallikrein, prothrombin, prothrombin degradation products, prothrombin fragment 1+2, Protein C, Protein S, thrombin, thrombin complexes, thrombin-antithrombin complexes, antithrombin, tissue factor, tissue plasminogen activator, and anti-cardiolipin antibody. Standard curves may be established utilizing droplets having standard concentrations of the target analyte. For example, a TAT or F1+2 standard curve may be established on the droplet actuator using concentrations of standard ranging from about 0.0 to about 240 ng/mL.

In some embodiments, accurate ELISA for prothrombin F1+2 may require the sample undergoing analysis to be substantially devoid of prothrombin and/or other interfering contaminants, such as F1, prothrombin and prothrombin-2. Antibodies directed against F1+2 may also detect the presence of any prothrombin within the solution. To solve this problem, it is useful to subject samples to incubation with magnetically responsive beads coated with anti-thrombin antibody. Anti-thrombin antibody-coated magnetically responsive beads will also bind prothrombin and clean up the sample. The beads may be removed using a magnetic field with sufficient magnetic force to remove the beads from the droplet, e.g., the beads may be pulled out of the droplet as the droplet is transported using electrode-mediated droplet operations through the magnetic field. Once prothrombin is removed from the sample, the ELISA assays for F1 and F2 may be performed on the residual supernatant. In another embodiment, low affinity antibodies may be used which allow for the assay of F1+2 in bodily fluids that also contain prothrombin, or other plasma proteins, such as the antibodies described in Ruiz et al., U.S. Pat. No. 6,541,275, entitled “Immunoassay for F1.2 Prothrombin Fragment,” granted on Apr. 1, 2003.

In certain embodiments, multiple samples (e.g., duplicates, triplicates, etc.) of known concentrations of prothrombin (e.g., range 40 to 1024 ng/ml) may be subjected to multi-station ELISA on the droplet actuator. For example, a droplet of standard or sample may be combined using droplet operations with a droplet of magnetically responsive beads coated with anti-thrombin antibody. The supernatant may then be separated from the beads and subjected to F1+2 ELISA using a droplet operations protocol at a second station to determine the presence of any residual prothrombin. The concentration of prothrombin may be increased gradually to determine the threshold concentration beyond which all of the anti-thrombin affinity sites become saturated and thereby result in spillover of residual prothrombin into the F1 and F2 immunoassays. If affinity binding fails to remove >99% of prothrombin from the sample using the first set of anti-thrombin antibody beads, the number of anti-thrombin antibody beads may be increased to bind a larger amount of prothrombin. Alternatively, the supernatant may subjected to a second thrombin cleaning pass using a second set of anti-thrombin antibody beads prior to F1+2 ELISA.

A standard curve may be generated on the droplet actuator for known concentrations of thrombin standard. Thrombin may be reconstituted in a buffer, such as Hepes-NaCl buffer containing 1% bovine serum albumin (BSA), e.g., at 10 different concentrations (range 5-500 ng/ml) using a droplet actuator serial dilution protocol. For example, a first reservoir may be loaded with a 500 ng thrombin solution, and 9 other reservoirs may be either pre-loaded with buffer or loaded with buffer, using droplet operations, from a large reservoir containing buffer on the droplet actuator. One or more droplets may be dispensed from the thrombin solution reservoir and transported into the first of the 9 buffer reservoirs. Sufficient droplets may be added to bring the first buffer reservoir to a desired thrombin concentration. Next, one or more droplets from the thrombin solution reservoir and/or the first buffer reservoir may be transported into the second buffer reservoir to bring the second buffer reservoir to a desired thrombin concentration. The process may be repeated with each subsequent buffer reservoir, using droplets from the other reservoirs until the desired concentration of thrombin is achieved in each of the 9 reservoirs. It will be appreciated that, depending on the concentration of thrombin desired, various steps in the process may be conducted in parallel or in reverse. For example, in one embodiment, one droplet of 500 ng thrombin solution is transported into a first buffer reservoir, two droplets into the next, three into the next, and so on. Moreover, different volumes of buffer may be loaded in each reservoir to facilitate a shorter serial dilution protocol. Between additions of thrombin droplets to buffer reservoirs, it is helpful to agitate the liquid in the reservoir to promote thorough mixing prior to dispensing a droplet destined for another buffer reservoir. Mixing can, for example, be achieved using vibration, such as by sonication or piezoelectric crystal vibration. A convenient approach to mixing involves repeatedly dispensing a droplet from a reservoir and adding the droplet back to the reservoir. In another approach, various electrode arrangements may be provided within the reservoir for transporting the droplet back and forth to promote mixing. Combinations of mixing approaches may also be used. Reagents for generating a standard curve are available from Technoclone Ltd., Vienna, Austria (Technothrombin® assay kit).

A fluorogenic substrate solution may be loaded into a reservoir on the droplet actuator. For example, the fluorogenic substrate solution may include 1 mM Z-Gly-Gly-Arg-AMC, with 15 mM CaCl₂ and LPI. Alternatively, the components of the fluorogenic substrate solution may be present on the droplet actuator and may be combined using droplet operations to yield the fluorogenic substrate solution. One or more droplets of fluorogenic substrate solution may be combined using droplet operations with one or more droplets of thrombin standard or thrombin sample. Fluorescence from the combined droplet may be measured using a suitable protocol. For example, fluorescence may be measured using continuous measurement over a time ranging from about 1,2,3,4,5,6,7,8 or 9 or more minutes to about 2,3,4,5,6,7,8,9 or 10 or more minutes or intermittently for a time period of up to 1 hour wherein the fluorescing droplet will move into and out of the field of view of the fluorimeter so that the fluorimeter is available for measurements on other droplets. Fluorescence may be measured at a suitable wavelength, e.g., 360 nm/460 nm [excitation/emission]. Miniature fluorometers may be used with interchangeable filters and dichroic mirrors for droplet actuator detection of enzymatic activity. An assay may, for example, be designed to provide excitation at 360 nm with a UV diode and a filter and dichroic beam splitter configured to collect emission at 460 nm with a field of view of ˜2 mm so that a droplet fits within it.

Droplet operations required for accomplishing the assays of the invention may be conducted on a droplet actuator. One or more of the assay droplet operations may be conducted on a droplet operations surface of a droplet actuator. One or more of the assay droplet operations may be conducted in a droplet operations gap of a droplet actuator. For example, certain droplet actuators will include a substrate, droplet operations electrodes associated with the substrate, one or more dielectric and/or hydrophobic layers atop the substrate and/or electrodes forming a droplet operations surface, and optionally, a top substrate separated from the droplet operations surface by a droplet operations gap. One or more reference electrodes may be provided on the top and/or bottom substrates and/or in the gap. One or more droplet operations of the assays of the invention may be electrode-mediated, e.g., electrowetting mediated or dielectrophoresis mediated or Coulombic force mediated. In some embodiments, the droplet actuator is provided as a portable device, permitting analysis at a point of sample collection. In other embodiments, it is provided as an in-line device in an extracorporeal circulation device. The device may produce an output which is interpreted by a user and used to guide treatment decisions such as the administration of coagulants and/or anticoagulants. The device may also be part of a system which automatically controls the administration of one or more therapies in response to the output.

Assays using coagulated blood samples and products of coagulation may employ any of a variety of suitable detection techniques. Examples of detection techniques are described in International Patent Application No. PCT/US 06/47486, filed on Dec. 11, 2006, entitled “Droplet-Based Biochemistry,” the entire disclosure of which is incorporated herein by reference. In one embodiment, the assays on whole blood samples make use of luminescence detection, such as chemiluminescence detection.

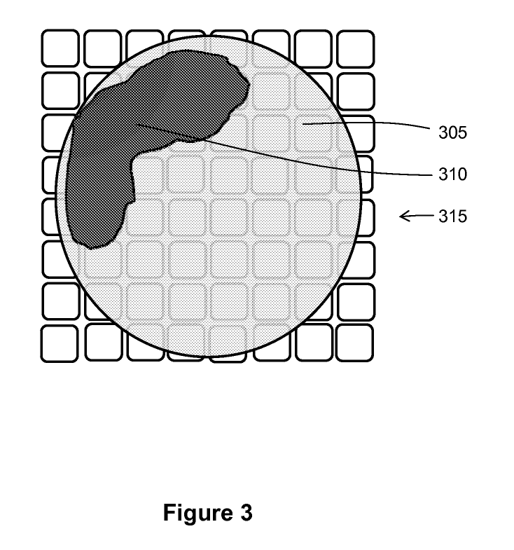

FIG. 3 illustrates an embodiment of the invention in which impedance detection is used to detect a coagulated region within a droplet. A droplet 305 including a coagulated region 310 is atop an array of electrodes 315. For example, coagulated region 310 may be a blood clot within a serum droplet 305. Electrodes in electrode array 315 may be activated together to function as a single electrode for the purposes of conducting certain droplet operations, such as droplet transport. Each electrode may be interrogated separately for their impedance. The impedance will differ at the electrodes where a clot is formed compared to where the serum is present. The percentage of droplet that is clotted can then be assessed by measuring the impedance across all the electrodes and calculating the electrodes that correspond to that of a clot. Further, a splitting operation may be effected based on the location of the coagulated portion 310 of droplet 305 in order to maximize the volume of serum obtained in a daughter droplet substantially lacking in coagulated material.

### 7.6 Systems

As will be appreciated by one of skill in the art, the invention may be embodied as a method, system, or computer program product. Accordingly, various aspects of the invention may take the form of hardware embodiments, software embodiments (including firmware, resident software, micro-code, etc.), or embodiments combining software and hardware aspects that may all generally be referred to herein as a “circuit,” “module” or “system.” Furthermore, the methods of the invention may take the form of a computer program product on a computer-usable storage medium having computer-usable program code embodied in the medium.

Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include some or all of the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Computer program code for carrying out operations of the invention may be written in an object oriented programming language such as Java, Smalltalk, C++ or the like. However, the computer program code for carrying out operations of the invention may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network (LAN) or a wide area network (WAN), or the connection may be made to an external computer (for example, through the Internet using an Internet Service Provider).

Certain aspects of invention are described with reference to various methods and method steps. It will be understood that each method step can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the methods.

The computer program instructions may also be stored in a computer-readable memory that can direct a computer or other programmable data processing apparatus to function in a particular manner, such that the instructions stored in the computer-readable memory produce an article of manufacture including instruction means which implement various aspects of the method steps.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing various functions/acts specified in the methods of the invention.

The detection system may comprise a fluorimeter, a luminometer, and a colorimeter. For example, thrombin generation could be measured through a functional assay which will be measured through a fluorimeter and the ELISA for thrombin could be measured through a luminometer to measure the chemiluminescence. In another embodiment, the detection system can comprise of a single detector such as just a fluorimeter where the functional assay for thrombin results in a fluorescent product with an emission at 460 nm and the ELISA for thrombin could also result in a fluorescent product with emission around 460 nm where a substrate such as 4-methylumbelliferyl-phosphate will be cleaved by the alkaline phosphatase conjugated to the secondary antibody to yield 4-methylumbelliferone which has emission around 460 nm.

### 8 EXAMPLES

The following examples are for the purpose of illustrating certain aspects or embodiments of the invention and are not intended to limit the scope of the invention.

### 8.1 Preparation of TAT and F1+2 Magnetically Responsive Beads

Rabbit anti-sheep IgG coated magnetically responsive beads (Isogen Lifescience, Ijsselstein, Netherlands) were reconstituted in coating buffer and incubated with either sheep anti-human thrombin, anti-prothrombin F1, or F2 antibodies (Affinity Biologicals, Ontario, Canada) as per manufacturer recommendations. Remaining IgG binding sites on the magnetically responsive beads were filled by incubating them with non-specific sheep antibodies. Beads were immobilized with a strong magnet and washed with PBS five times, and resuspended in buffered protein base (2 mg/ml).

### 8.2 Droplet Actuator ELISA Assay

The sequence described above may be translated into droplet operations on the droplet actuator as follows. Briefly, human thrombin-antithrombin complex and prothrombin fragments 1+2 may be reconstituted in buffered protein base at the same concentrations outlined above and loaded onto the droplet actuator. The sequence for performing the immunoassays may remain the same as above; however, the volumes of reagents and samples may be scaled down 50-fold.

Magnetically responsive beads (2 mg/ml) may be prepared as described above, and one droplet (320 nL) may be distributed to separate electrodes. One droplet (320 nL) of each sample may be transported to the corresponding electrode containing magnetically responsive beads, and allowed to incubate for 2 minutes. One droplet of wash buffer may be added to the solution to create a 3×(˜1 μL) droplet. Magnetically responsive beads may be immobilized using a magnet, and a 1× droplet may be split off from the magnetically responsive bead electrode and discarded. This process of wash buffer addition and removal may be repeated five times to achieve serial dilution of immobilized magnetically responsive beads. One droplet (320 nL) of secondary antibody conjugated to peroxidase may be mixed with the magnetically responsive bead droplet at each electrode, and allowed to incubate for 2 minutes with the magnet off. The beads may once again be immobilized, and washed by sequential mixing and splitting of buffer solution as described above. Excess wash buffer solution may be disposed. 1× droplet of magnetically responsive beads with secondary antibody may be transported to the detection zone, where 1× droplet of Lumigen PS-atto chemiluminescence substrate may be added. Chemiluminescence may be measured using a photomultiplier tube (PMT). A droplet of the substrate and a droplet of activated sample droplet are mixed on the droplet actuator and monitored at the fluorimeter for fluorescence initiated by the generated thrombin.

### 8.3 Rate of Thrombin Generation

Whole blood may be collected in corn trypsin inhibitor (CTI) or an equivalent to prevent premature contact pathway initiation of the coagulation system. For testing purposes, blood collected in sodium citrate may be utilized. 100 μL sample may be loaded into a collecting chamber containing CTI, CaCl₂, and relipidated TF to create final concentrations of 32 μg/mL CTI and 40 pmol/L tissue factor, 15 mM CaCl₂ and 80 nmol/L PCPS.

All reagents may be loaded into reagent loading reservoirs on the droplet actuator. The protocol may be executed and controlled by software. Sample may be loaded into a sample loading reservoir. 24 aliquots (each 1× droplet, or ˜320 nL) may be dispensed using droplet operations from the sample loading reservoir and positioned on assigned electrodes and coagulation is initiated with a procoagulant droplet. The coagulation may be quenched at different times in each aliquot droplet by combining the aliquot droplet with a droplet of quenching solution. For example, the quenching solution may include EDTA (50 mM), 20 mM benzamidine-HCl in HEPES-buffered saline (HBS), and 10 mM D-Phenylalanyl-L-prolyl-L-arginine chloromethyl ketone (FPRck in 10mM HCl). The first aliquot droplet is quenched 2 minutes following activation, and subsequent droplets are quenched at 2 minute time intervals over the course 48 minutes.

After quenching, each sample may be analyzed by sandwich ELISA on the droplet actuator to detect the formation of TAT complexes and F1+2 as follows. The quenched droplet (3×) may be transported to its assigned TAT station (1× droplet magnetically responsive beads (2 mg/ml) coated with anti-thrombin antibody), and allowed to incubate for 2 minutes. Magnetically responsive beads may be immobilized by the on the droplet actuator magnet, and 1× supernatant is split off from the TAT station.

This supernatant may be transported using droplet operations to the F1+2 magnetically responsive bead station to perform the F1+2 ELISA as described below. Immobilized magnetically responsive beads at the TAT station may be washed with serial addition and removal of wash buffer droplets, and all removed droplets may be transported using droplet operations to the F1+2 ELISA station.

1× droplet conjugated secondary antibody to F1+2 may be incubated with the TAT station beads for 2 minutes with the magnet released. The beads will once again be immobilized and may be washed using a droplet washing protocol. Excess wash buffer solution may be transported to a waste reservoir. 1× droplet TAT beads with secondary antibody may be transported to the detection zone, where 1× droplet Lumigen PS-atto chemiluminescence substrate may be added. Chemiluminescence may be measured with the PMT.

In parallel with the ELISA at the TAT station, F1+2 ELISA may be performed at the second station. The sample for this station may be the supernatant removed from the TAT station if prothrombin or thrombin has to be removed in the earlier step. Following 2 minute incubation, the F1+2 beads may be washed by serial dilutions with wash buffer (about 10 times). Magnetically responsive beads may be incubated with conjugated secondary antibody against F1 and F2, and washed again (10 times). The droplet of magnetically responsive beads may be transported to the detection zone, and mixed with 1× droplet of substrate. Chemiluminescence may be measured by the PMT.

The 24 measurements of luminescence may be used to calculate TAT complex and F1+2 levels, which are plotted as a function of time. The generated curve may be analyzed to determine the lag time to thrombin generation and the total amount of TAT complex or F1+2 generation. The slope of the best-fit function may be used to determine the rate of thrombin generation, and the peak thrombin generation rate may be identified.

### 8.4 Assay by Enzymatic Cleavage of Fluorogenic Substrate

Demonstrations are provided of assay based on the cleavage of a fluorescent substrate by thrombin on a droplet actuator using 320 nanoliter droplet of sample. Thrombin generation was determined by adapting a commercially available assay from Technoclone (Technothrombin assay) for use on a droplet actuator. All reagents and samples were reconstituted according to the manufacturer's instructions. Thrombin generation was measured on three control samples using a substrate/reagent mixture containing a low concentration of phospholipid micelles and ˜5 pM tissue factor (assay Reagent C, High). The control samples were normal human plasma (C1-360nM peak thrombin), human plasma with increased thrombin generation (C2-472 nM peak thrombin) and human plasma with decreased thrombin generation (C3-69 nM peak thrombin).

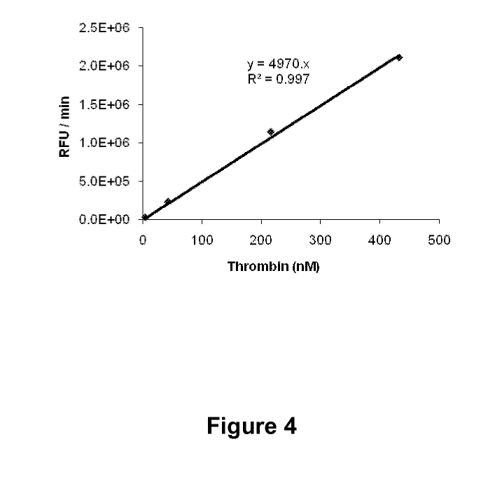

The Technoclone thrombin generation assay was adapted to our digital microfluidic droplet actuators in the following manner All of the on-actuator experiments were performed at room temperature. Thrombin standards were prepared off-actuator by serial dilution of thrombin to make four thrombin standards at concentrations of 4.3, 43.3, 216.3 and 432.5 nM. Droplet operations, including dispensing, transport, mixing, incubating, and disposing, were performed using software control of electrodes on a droplet actuator. The 1× droplets were about 320 nanoliters. A thrombin standard curve was produced on-actuator by mixing one droplet of a thrombin standard with one droplet of the thrombin fluorogenic substrate ZGGR-AMC to initiate the reaction. The fluorescence of the merged droplets was measured at Ex 360 nm/Em 440 nm at 30 second intervals for 10 minutes. For each thrombin standard, the average ΔRFU/minute was calculated and plotted against the concentration of thrombin. The thrombin standard curve generated on-actuator is shown in FIG. 4.

The human plasma control samples were tested on-actuator in a manner analogous to the testing of thrombin standards. One droplet of a plasma control sample was merged with one droplet of the thrombin substrate ZGGR-AMC to initiate the reaction and the increase in fluorescent signal recorded as described above. The control samples were read for a total of 70 minutes at 1 minute intervals. A continuous increase in fluorescence with time was observed for the three human plasma control samples after merging the samples with the substrate on-actuator. The amount of fluorescent signal corresponded to the quantity of thrombin in the plasma samples.

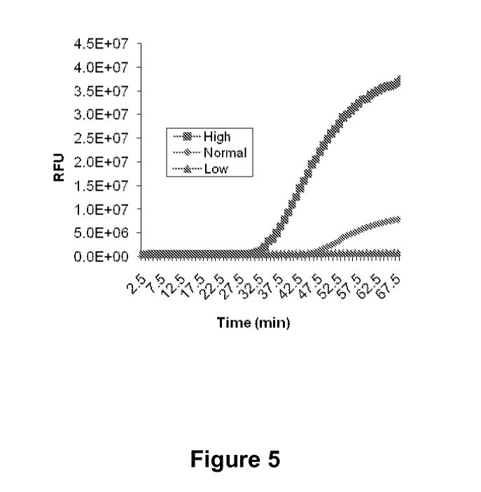

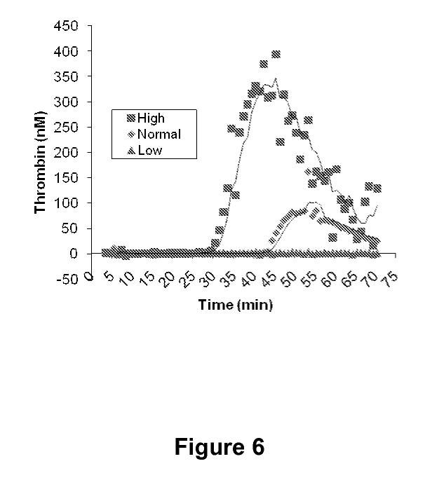

FIG. 5 shows kinetic fluorescence curves from high, normal, and low plasma samples for on-actuator activation of thrombin generation. FIG. 6 shows rate of fluorescence (from FIG. 5) fit into the standard curve to demonstrate thrombin generation curves produced on-actuator.

After completion of the reaction, the ARFU/minute was converted to nM thrombin for each control sample using the thrombin standard curve and replotted against time. These curves shown in FIG. 6 depict the actual effective thrombin concentration for all three plasma control samples. It was not attempted to remove the noise. The line plot in FIG. 6 utilizes a moving average fit to the data to smooth out the observed noise. The general overall shape of the thrombin generation curves generated on-actuator for the three control plasma samples are as expected with a lag phase, an initial slope, thrombin potential, peak time, peak thrombin value, and decay. However, each sample showed an extended lag phase of at least thirty minutes and a lower than expected observed peak thrombin value on-actuator. These discrepancies could be attributed to performing the assays on droplet actuator at 25° C. instead of the manufacturer's recommendation of 37° C. On-actuator thermal control could remedy the observed lower values for peak thrombin on-actuator and the prolonged lag phase.

### 8.5 Multiplexed Thrombophilia Panel

A fully automated multiplexed ELISA for Proteins C and S, Factor VIII, homocysteine, antithrombin III, and anticardiolipin antibody can be translated onto the digital microfluidic platform with high fidelity. The on-actuator multiplexed ELISA can be performed with smaller sample size and less reagents. Multiplexed immunoassays for the thrombophilia panel will have low CVs (coefficient of variance) and high levels of reproducibility.

Dilutions of Proteins C and S, Factor VIII, homocysteine, antithrombin III, and anticardiolipin antibody stock solutions will be made to create solutions ranging in concentration from 0.1% to 10 fold increase from normal physiologic values (see Table 1). ELISA will be performed on the digital microfluidic cartridges to generate the coagulation factor standard curves. All the primary capture antibodies will be conjugated to carboxylated-magnetic beads and secondary antibodies, where not available, will be conjugated with alkaline phosphatase. All the reagents will be loaded simultaneously onto the actuator along with the standard solutions. All assays will be performed in triplicates, and data obtained on-actuator will be analyzed to obtain correlation coefficients.

TABLE 1
Thrombophilia panel reference range

| Factor | Physiologic reference range | |
|---|---|---|
| Antithrombin III | 170-390 | mg/L |
| Protein C | 3 | mg/L |
| Protein S | 0.5-1.17 | U/ml |
| Factor VIII | 0.5-1.58 | iU/ml |
| Anticardiolipin antibody | <15 | units |
| Homocysteine | <7 | micromol/L |

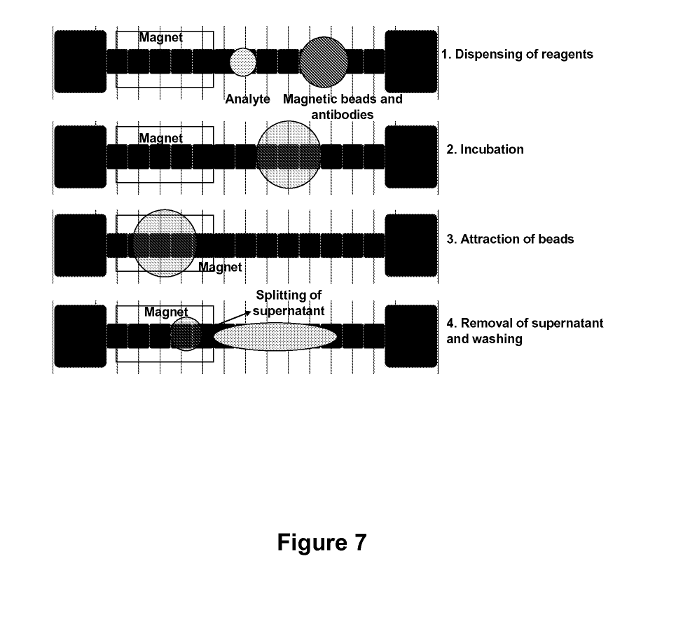

On-actuator Assay Development: Individual immunoassays for protein C and S, Factor VIII, homocysteine, Antithrombin III, and anticardiolipin antibody may be provided. FIG. 7, described below, outlines the on-actuator methodology for ELISA. Reagent Optimization—Optimize concentrations of beads, immobilized capture antibody, and secondary antibody for each immunoassay. Protocol Optimization—Optimize incubation protocols and times and the number of washes. Utilize other surfactants, if needed, to reduce background if any. The surfaces of the actuators are protected from the droplets by a thin immiscible filler fluid therefore there was no observation of any non-specific binding to the surfaces and even if there were non-specific adsorption, it has been found that it can be cleared by exposing the surface to one or more “wash” droplets.

Standard Curve: Data Analysis—The accuracy of a quantitative immunoassay depends on the quality of the standard curves. At least 8 different concentrations of the standards will be usedto generate a calibration curve ranging from 0.1% of normal to 10 fold normal physiologic values. The data will be fit using a 5-parameter logistic (5-PL) equation, which is more robust, least influenced by anomalous data, provides better interpolation of unknowns at both low and high concentrations, and particularly suited for fitting immunoassays. The 5-PL equation is described below:

$$y = d + \frac{a - d}{{[ 1 + {( \frac{x}{c} )}^{b} ]}^{g}}$$

where y is the measured signal, x is the analyte concentration, a is the estimated response at zero concentration, b is the slope of the tangent at midpoint, c is the midrange concentration or midpoint (corrected for non-specific binding), d is the estimated response at infinite concentration, and g is the asymmetry factor.

### 8.6 Multiplexed ELISA Thrombophilia Panel on Reconstituted Whole Blood

Once assay performance has been assessed and standard curves established in non-blood medium, the accuracy of the multiplexed ELISA in whole blood samples will be tested by repeating above experiments in reconstituted whole blood. This allows examination of any interference between specific antigens or antibodies and the solid or plasma phase of whole blood. In previous experiments examining other cardiac markers (see section D), such interaction has been found to be negligible.

Immunodepleted plasma (Aniara Corp., Mason, Ohio) will be obtained that is immunodepleted of all thrombophilic factors (protein C and S, Factor VIII, homocysteine, and Antithrombin III antigens, and anticardiolipin antibody). Whole blood will be reconstituted by addition of washed red blood cells. Corresponding antigen (protein C and S, Antithrombin III, Factor VIII, homocysteine) or antibody (anticardiolipin antibody, Aniara) will be added in incremental doses to study a wide range of antigen or antibody profile. From each sample, about 3 μL will be utilized for on-actuator experiments.

Generation of Standard Curve: Multiplexed calibration standards will be created by combining the appropriate standard concentration for each analyte into one solution. The multiplexed protocol will be run on each combined standard on 8 separate formulations of reconstituted whole blood.

Calibration Methodology. 8 samples will be reconstituted consisting of varying concentrations of each of the analytes that spans the range. The multiplex assay will be performed on each of the 8 samples in triplicate (three different cartridges per sample). On each cartridge, the full set of calibration standards prepared will be run as described above in addition to a negative control (immunodepleted whole blood). The concentration of each analyte in each sample will be calculated with 3 different methods: 1) using the calibration curve generated with the on-cartridge calibrators; 2) using the mid level standard to adjust the reference curve generated and 3) using a low and a high standard to do a two point calibration of the external reference curve generated. The concentrations determined by each method will be compared to calculated concentrations to determine the method that provides the best fit and to determine our calibration strategy.

In alternative approaches, thrombophilia ELISA panel may be performed on PRP or PPP obtained by centrifugation of whole blood. Whereas performing the ELISA on plasma is considered acceptable, it would be preferable to accomplish whole blood assay which requires less sample processing prior to testing.

### 8.7 On-Cartridge ELISA

Samples and reagents will be loaded onto the microfluidic actuator. To perform coagulation factor ELISA, 1 droplet (320 nL) of each sample and 1 droplet of reagent, containing magnetic beads coated with primary antibody against the specific antigen or antibody to be tested, blocking antibodies, and alkaline phosphatase-labeled secondary antibody, will be transported, mixed, and allowed to incubate for 2 minutes. After incubation, several droplets of wash buffer will be added to the incubated magnetic beads. Magnetic beads will be immobilized by the on-actuator magnet, and the supernatant will be split off as droplets and discarded. This process of wash buffer addition and removal will be repeated five times to achieve serial dilution of immobilized magnetic beads (FIG. 7). After washing, the droplet containing magnetic beads with sandwich of primary antibody, antigen, and secondary antibody will be transported to a detection zone, where it will be mixed with a droplet of Lumigen APS-5 (chemiluminescence substrate) and chemiluminescence will be measured with a photo multiplier tube (PMT). The resultant chemiluminescence will be used to determine the concentration of the specific coagulation factor. In one embodiment, 2 immunoassays can be simultaneously performed on 12 different samples yielding 24 immunoassays in one set of operations. This will be repeated thrice to perform all the 6 immunoassays on up to 12 samples, yielding a total of 72 immunoassays on a single cartridge. In one embodiment all the sample droplets are transported along sample lanes in an x axis while staying in their respective pathways to avoid cross contamination, while the reagent droplets are dispensed from reservoirs on the top and bottom ends of the layout and transported into the sample lanes along a generally perpendicular y axis. The sequence of operations shown in FIG. 7 is carried out, and each droplet with antibody-antigen-antibody sandwich on magnetic beads is mixed with a substrate droplet and transported through a fixed detection spot which is coupled to a PMT.

### 9 CONCLUDING REMARKS

The foregoing description of embodiments of the invention and examples refers to the accompanying drawings, which illustrate specific embodiments of the invention. Other embodiments having different structures and operations do not depart from the scope of the invention. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the invention may be changed without departing from the scope of the invention. Furthermore, the foregoing description is for the purpose of illustration only, and not for the purpose of limitation, as the invention is defined by the claims as set forth hereinafter.

1. A method of effecting coagulation in source droplet, the method comprising:
(a) providing a liquid filler fluid;
(b) providing in the liquid filler fluid a source droplet comprising one or more coagulatable substances;
(c) treating in the liquid filler fluid the source droplet to effect coagulation of the one or more coagulatable substances to yield a coagulated droplet in the liquid filler fluid comprising a coagulated portion and supernatant.

2. The method of claim 1 wherein the source droplet comprises a biological fluid.

3. The method of claim 1 wherein the biological fluid comprises a blood sample.

4. The method of claim 3 wherein the blood sample comprises whole blood.

5. The method of claim 3 wherein the blood sample consists essentially of whole blood.

6. The method of claim 3 wherein the blood sample consists of whole blood.

7. The method of claim 3 wherein the blood sample comprises one or more natural blood components.

8. The method of claim 1 wherein the blood sample comprises one or more artificial blood components.

9. The method of claim 1 wherein the blood sample comprises one or more anticoagulants.

10. The method of claim 9 wherein the anticoagulant is selected from the group consisting of coumarines, vitamin K antagonists, acenocoumarol, phenprocoumon, brodifacoum, phenindione, heparins, low molecular weight heparin, synthetic pentasaccharide inhibitors of Factor Xa, and thrombin inhibitors.

11. The method of claim 8 wherein the one or more artificial blood components comprise one or more artificial platelet components.

12. The method of claim 8 wherein the one or more artificial blood components comprise one or more artificial oxygen carriers.

13. The method of claim 1 wherein the source droplet comprises a milk sample.

14. The method of claim 1 wherein the source droplet comprises a plant sample.

15. The method of claim 1 wherein the source droplet comprises coagulatable beads.

16. The method of claim 1 wherein step 1(c) comprises combining the sample droplet with a procoagulant droplet comprising a procoagulant.

17. The method of claim 1 wherein step 1(c) comprises contacting the sample droplet with a procoagulant.

18. The method of claim 1 wherein step 1(c) comprises incubating the sample droplet for a period of time sufficient to permit coagulation.

19. The method of claim 1 wherein step 1(c) comprises maintaining the sample droplet in a substantially stationary position for a period of time sufficient to permit coagulation.

20. The method of claim 1 wherein step 1(c) comprises heating the sample droplet.

21. The method of claim 1 wherein step 1(c) comprises cooling the sample droplet.

22. The method of claim 1 wherein step 1(c) is accomplished in the presence of an electrical field.

23. The method of claim 1 further comprising conducting an assay using the coagulated droplet as input.

24. The method of claim 1 wherein the method is effected using droplet operations on a droplet actuator.

25. The method of claim 1 wherein the method is effected using droplet operations in a droplet operations gap on a droplet actuator.

26. The method of claim 1 wherein the liquid filler fluid comprises a silicone oil, a carbon oil, and/or a fluorinated oil.

27. The method of claim 1 wherein the liquid filler fluid has a viscosity ranging from about 1 to about 3 cSt.

28. The method of claim 1 wherein the liquid filler fluid is doped with a surfactant.

29. The method of claim 28 wherein the surfactant comprises a linoleic acid based surfactant composition.

30-136. (canceled)
